(19) United States
(12) Patent Application Publication
(10) Pub. No.: US 2007/0185374 A1
(43) Pub. Date: Aug. 9, 2007

(54) Two-way adjustable implant

(75) Inventors: George F. Kick, Casa Grande, AZ (US); Jay A. Lenker, Laguna Beach, CA (US)

Correspondence Address: MCDERMOTT WILL & EMERY LLP, 18191 VON KARMAN AVE., SUITE 500, IRVINE, CA, 92612-7108, US

(73) Assignee: Ellipse Technologies, Inc.

(21) Appl. No.: 11/654,068

(22) Filed: Jan. 16, 2007

Related U.S. Application Data
(60) Provisional application No. 60/759,672, filed on Jan. 17, 2006.

(51) Int. Cl. A61F 2/00

(52) U.S. Cl. 600/37

(57) ABSTRACT

An adjustable implant configured to be implanted within or at least partially around an outer surface of a stomach or esophagus is described. The adjustable implant includes a ratchet. The implant further includes an elongate band comprising a shape-memory material, wherein a first end and a second end of the elongate band are configured to couple to the ratchet, such that the band and the ratchet form an assembly having a loop configuration. Activation of the shape-memory material adjusts the band from a first length to a second length as the ratchet permits movement in a first direction of the first end relative to the second end, changing a circumference of the loop configuration.

### CROSS REFERENCE TO RELATED APPLICATIONS

This application claims the benefit under 35 U.S.C. § 119(e) of U.S. Provisional Patent Application No. 60/759,672, filed on Jan. 17, 2006, and titled “TWO-WAY ADJUSTABLE IMPLANT,” the entirety of which is hereby incorporated by reference.

### BACKGROUND OF THE INVENTION

1. Field of the Invention

The present invention relates to devices and methods for dynamically restricting the capacity of the stomach using an implant or implants within or around the outside of the stomach and externally or internally activating the implant(s) to induce a change in shape and/or size of the implant(s).

2. Description of the Related Art

Obesity is a common disease of unknown etiology. It is a chronic, multifactorial disease that develops from an integration of genetic, environmental, social, behavioral, physiological, metabolic, neuron-endocrine and psychological elements. This disease is considered a cause or comorbidity to such conditions as GERD, high blood pressure, elevated cholesterol, diabetes, sleep apnea, mobility and orthopedic deterioration, and other consequences, including those limiting social and self image and those affecting the ability to perform certain everyday tasks. Since traditional weight loss techniques, such as diet, drugs, exercise, etc., are ineffective with many of these patients, surgery is often the only viable alternative.

Body Mass Index (BMI) is the most common method used to define the obese patient. This measurement is obtained by taking a persons weight in Kilograms (Kg) and dividing by the square of height in meters. Based on policies set forth by the United States National Institutes of Health (NIH), BMI is used to characterize the degree of excess weight. These categories are listed in Table 1. Presently, only those people with a BMI of 35 or greater qualify for surgical intervention based on NIH policy.

TABLE 1
Risk of Associated Disease According to BMI and Waist Size

| | | Disease Risk | Disease Risk |
|---|---|---|---|
| | | Waist ≦40 in. | Waist >40 in. |
| | Weight | (men) or 35 in. | (men) or 35 in. |
| BMI | Classification | (women) | (women) |
| 18.5 or less | Underweight | — | N/A |
| 18.5-24.9 | Normal | — | N/A |
| 25.0-29.9 | Overweight | Increased | High |
| 30.0-34.9 | Obese | High | Very High |
| 35.0-39.9 | Severely Obese | Very High | Very High |
| 40 or greater | Extremely Obese | Extremely High | Extremely High |

In the United States, more than 30% of the population is obese as defined in Table 1, including men, women, and children. There are more than 15 million Americans (5.5%) who are morbidly obese. The number of obese children is growing at an alarmingly fast rate. Surgical treatments for obesity continue to be a strong focus of research due to their high level of effectiveness although no treatment is considered ideal. Much work continues to be needed before a widely acceptable solution can be expected.

Surgical weight loss (bariatric) procedures are designed to restrict weight gain by either limiting caloric intake by restricting effective stomach size or by malabsorption, which is reducing the intestine's ability to absorb nutrition. Many surgeons offer their patients a combined procedure that includes a restrictive and malabsorption material. These procedures are irreversible and rely on a surgeon's judgment to estimate the final size of the new restrictive stomach as well as the remaining small intestine length to provide adequate nutrition for optimal weight loss and management for the patient's lifetime.

Presently, bariatric procedures can be performed by open or laparoscopic surgery. Open surgery typically requires a 10 day hospitalization and a prolonged recovery period with a commensurate loss of productivity. Laparoscopic procedures have reduced in-hospital stay to 3 days, followed by a 3 week at-home recovery. These procedures can even be performed as an outpatient procedure. Laparoscopic procedures have reduced cost considerably, making the minimally invasive laparoscopic procedure available to more patients. In 2000, there were 30,000 bariatric procedures performed, while in 2003, over 90,000 procedures were reported.

One common obesity surgery is the Roux-en-Y gastric bypass (often known only as a “gastric bypass”). During this type of operation, the surgeon permanently changes the shape of the stomach by surgically reducing (cutting or stapling) its size to create an egg-sized gastric pouch or “new stomach.” The rest of the stomach is then divided and separated from this new stomach pouch, greatly reducing the amount of food that can be consumed after surgery. In addition to reducing the actual size of the stomach, a significant portion of the digestive tract is bypassed and the new stomach pouch is reconnected directly to the bypassed segment of small intestine. This operation, therefore, is both a restrictive and malabsorptive procedure, because it limits the amount of food that one can eat and the amount of calories and nutrition that are absorbed or digested by the body. Once completed, gastric bypass surgery is essentially irreversible. Some of the major risks associated with the Roux-en-Y Gastric Bypass procedure include: bleeding, infection, pulmonary embolus, anastomotic stricture or leak, anemia, ulcer, hernia, gastric distention, bowel obstruction and death.

Another common obesity surgery is known as vertical banded gastroplasty (“VBG”), or “stomach stapling.” In a gastroplasty procedure, the surgeon staples the upper stomach to create a small, thumb-sized stomach pouch, reducing the quantity of food that the stomach can hold to about 1-2 ounces. The outlet of this pouch is then restricted by a band that significantly slows the emptying of the pouch to the lower part of the stomach. Aside from the creation of a small stomach pouch, there is no other significant change made to the gastrointestinal tract. So while the amount of food the stomach can contain is reduced, the stomach continues to digest nutrients and calories in a normal way. This procedure is purely restrictive; there is no malabsorptive effect. Following this operation, many patients have reported feeling full but not satisfied after eating a small amount of food. As a result, some patients have attempted to get around this effect by eating more or by eating gradually all day long. These practices can result in vomiting, tearing of the staple line, or simply reduced weight loss. Major risks associated with VBG include: unsatisfactory weight loss or weight regain, vomiting, band erosion, band slippage, breakdown of staple line, anastomotic leak, and intestinal obstruction.

A third procedure, the Duodenal Switch, is less common. It is a modification of the biliopancreatic diversion or “Scopinaro procedure.” While this procedure is considered by many to be the most powerful weight loss operation currently available, it is also accompanied by significant long-term nutritional deficiencies in some patients. Many surgeons have stopped performing this procedure due to the serious associated nutritional risks.

In the Duodenal Switch procedure, the surgeon removes about 80% of the stomach, leaving a very small new stomach pouch. The beginning portion of the small intestine is then removed, and the severed end portions of the small intestine are connected to one another near the end of the small intestine and the beginning of the large intestine or colon. Through this procedure a large portion of the intestinal tract is bypassed so that the digestive enzymes (bile and pancreatic juices) are diverted away from the food stream until very late in the passage through the intestine. The effect of this procedure is that only a small portion of the total calories that are consumed are actually digested or absorbed. This irreversible procedure, therefore, is both restrictive (the capacity of the stomach is greatly reduced) and malabsorptive (the digestive tract is shortened, severely limiting absorption of calories and nutrition). Because of the very significant malabsorptive material of this operation, patients must strictly adhere to dietary instructions including taking daily vitamin supplements, consuming sufficient protein and limiting fat intake. Some patients also experience frequent large bowel movements, which have a strong odor. The major risks associated with the Duodenal Switch are: bleeding, infection, pulmonary embolus, loss of too much weight, vitamin deficiency, protein malnutrition, anastomotic leak or stricture, bowel obstruction, hernia, nausea/vomiting, heartburn, food intolerances, kidney stone or gallstone formation, severe diarrhea and death.

One relatively new and less invasive form of bariatric surgery is Adjustable Gastric Banding. Through this procedure the surgeon places a band around an upper part of the stomach to divide the stomach into two parts, including a small pouch in the upper part of the stomach. The small upper stomach pouch can only hold a small amount of food. The remainder of the stomach lies below the band. The two parts are connected by means of a small opening called a stoma. Risks associated with Gastric Banding are significantly less than other forms of bariatric surgery, since this surgery does not involve opening of the gastric cavity. There is no cutting, stapling or bypassing.

It has been found that the volume of the small upper stomach pouch above the band increases in size up to ten times after operation. Therefore the pouch volume during surgery needs to be very small, approximately 7 ml. To enable the patient to feed the stomach with sufficient nutrition immediately after an operation considering such a small gastric pouch, the stoma initially needs to be relatively large and later needs to be substantially reduced, as the pouch volume increases. To be able to achieve a significant range of adjustment of the band, the cavity in the band has to be relatively large and is defined by a thin flexible wall, normally made of silicone material. Furthermore, the size of the stoma opening has to be gradually reduced during the first year after surgery as the gastric pouch increases in size. Reduction of the stoma opening is commonly achieved by adding liquid to the cavity of the band via an injection port to expand the band radially inwardly.

A great disadvantage of repeatedly injecting liquid via the injection port is the increased risk of the patient getting an infection in the body area surrounding the injection port. If such an infection occurs the injection port has to be surgically removed from the patient. Moreover, such an infection might be spread along the tube interconnecting the injection port and the band to the stomach, causing even more serious complications. Thus, the stomach might be infected where it is in contact with the band, which might result in the band migrating through the wall of the stomach. Also, it is uncomfortable for the patient when the necessary, often many, post-operation adjustments of the stoma opening are carried out using an injection needle penetrating the skin of the patient into the injection port.

It may happen that the patient swallows pieces of food too large to pass through the restricted stoma opening. If that occurs the patient has to visit a doctor who can remove the food pieces, if the band design so permits, by withdrawing some liquid from the band to enlarge the stoma opening to allow the food pieces to pass the stoma. The doctor then has to add liquid to the band in order to regain the restricted stoma opening. Again, these measures require the use of an injection needle penetrating the skin of the patient, which is uncomfortable for the patient, and can sometimes be the cause of infection, thus risking the long-term viability of the implant.

The LAP-BAND® Adjustable Gastric Banding System (Inamed) is a product used in the Adjustable Gastric Banding procedure. The LAP-BAND® system, includes a silicone band, which is essentially an annular-shaped balloon. The surgeon places the silicone band around the upper part of the stomach. The LAP-BAND® system further includes a port that is placed under the skin, and tubing that provides fluid communication between the port and the band. A physician can inflate the band by injecting a fluid (such as saline) into the band through the port. As the band inflates, the size of the stoma shrinks, thus further limiting the rate at which food can pass from the upper stomach pouch to the lower part of the stomach. The physician can also deflate the band, and thereby increase the size of the stoma, by withdrawing the fluid from the band through the port. The physician inflates and deflates the band by piercing the port, through the skin, with a fine-gauge needle. There is often ambiguous feedback to the physician between the amount injected and the restriction the patient feels during the adjustment procedure, such as when swallowing a bolus of liquid to test the stoma. In addition, a change of as little as 0.5 ml or less can sometimes make a difference between too much restriction and the correct amount of restriction.

The lower esophageal sphincter (LES) is a ring of increased thickness in the circular, smooth muscle layer of the esophagus. At rest, the lower esophageal sphincter maintains a high-pressure zone between 15 and 30 millimeters (mm) Hg above intragastric pressures. The lower esophageal sphincter relaxes before the esophagus contracts, and allows food to pass through to the stomach. After food passes into the stomach, the sphincter constricts to prevent the contents from regurgitating into the esophagus. The resting tone of the LES is maintained by myogenic (muscular) and neurogenic (nerve) mechanisms. The release of acetylcholine by nerves maintains or increases lower esophageal sphincter tone. It is also affected by different reflex mechanisms, physiological alterations, and ingested substances. The release of nitric oxide by nerves relaxes the lower esophageal sphincter in response to swallowing, although transient lower esophageal sphincter relaxations may also manifest independently of swallowing. This relaxation is often associated with transient gastroesophageal reflux in normal people.

Gastroesophageal reflux disease, commonly known as GERD, results from incompetence of the lower esophageal sphincter, located just above the stomach in the lower part of the esophagus. Acidic stomach fluids may flow retrograde across the incompetent lower esophageal sphincter into the esophagus. The esophagus, unlike the stomach, is not capable of handling highly acidic contents so the condition results in the symptoms of heartburn, chest pain, cough, difficulty swallowing, or regurgitation. These episodes can ultimately lead to injury of the esophagus, oral cavity, the trachea, and other pulmonary structures. GERD affects a large proportion of the population and mild cases can be treated with lifestyle modifications and pharmaceutical therapy. Patients, who are resistant, or refractory, to pharmaceutical therapy or lifestyle changes are candidates for surgical repair of the lower esophageal sphincter. The most common surgical repair, called fundoplication surgery, generally involves manipulating the diaphragm, wrapping the upper portion of the stomach, the fundus, around the lower esophageal sphincter, thus tightening the sphincter, and reducing the circumference of the sphincter so as to eliminate the incompetence. The hiatus, or opening in the diaphragm is reduced in size and secured with 2 to 3 sutures to prevent the fundoplication from migrating into the chest cavity. The repair can be attempted through open surgery, laparoscopic surgery, or an endoscopic, or endoluminal, approach by way of the throat and the esophagus. The open surgical repair procedure, most commonly a Nissen fundoplication, is effective but entails a substantial insult to the abdominal tissues, a risk of anesthesia-related iatrogenic injury, a 7 to 10 day hospital stay, and a 6 to 12 week recovery time, at home. The open surgical procedure is performed through a large incision in the middle of the abdomen, extending from just below the ribs to the umbilicus (belly button).

Endoscopic techniques for the treatment of GERD have been developed. Laparoscopic repair of GERD has the promise of a high success rate, currently 90% or greater, and a relatively short recovery period due to minimal tissue trauma. Laparoscopic Nissen fundoplication procedures have reduced the hospital stay to an average of 3 days with a 3-week recovery period at home. Another type of laparoscopic procedure involves the application of radio-frequency waves to the lower part of the esophagus just above the sphincter. The waves cause damage to the tissue beneath the esophageal lining and a scar (fibrosis) forms. The scar shrinks and pulls on the surrounding tissue, thereby tightening the sphincter and the area above it. These radio-frequency waves can also be used to create a controlled neurogenic defect, which may negate inappropriate relaxation of the LES. A third type of endoscopic treatment involves the injection of material or devices into the esophageal wall in the area of the lower esophageal sphincter. This increases the pressure in the lower esophageal sphincter and prevents reflux.

One laparoscopic technique that appears to show promise for GERD therapy involves approaching the esophageal sphincter from the outside, using laparoscopic surgical techniques, and performing a circumference reducing tightening of the sphincter by placement of an adjustable band such that it surrounds the sphincter. However, this procedure still requires surgery, which is more invasive than if an endogastric transluminal procedure were performed through the lumen of the esophagus or stomach, such as via the mouth. Furthermore, the necessity to provide for future adjustment in the band also requires some surgical access and this adjustment would be more easily made via a transluminal approach.

Evidence indicates that up to 36% of otherwise healthy Americans suffer from heartburn at least once a month, and that 7% experience heartburn as often as once a day. It has been estimated that approximately 1-2% of the adult population suffers from GERD, based on objective measures such as endoscopic or histological examinations. The incidence of GERD increases markedly after the age of 40, and it is not uncommon for patients experiencing symptoms to wait years before seeking medical treatment.

### SUMMARY OF THE INVENTION

Thus, it would be advantageous to develop systems and methods for placing an implant in or around a portion of a mammalian gut, such that the implant may be implanted and then noninvasively adjusted within the body of a patient. As used herein, the term “gut” refers to the whole alimentary tract, from mouth to anus, of a animal, or to any part thereof. An implant, an external adjustment system, and a method of use are provided according to embodiments of the inventions.

In certain embodiments, an adjustable implant is disclosed. The implant comprises a ratchet. The implant further comprises an elongate band comprising a shape-memory material, wherein a first end and a second end of the elongate band are configured to couple to the ratchet, such that the band and the ratchet form an assembly having a loop configuration. Activation of the shape-memory material adjusts the band from a first length to a second length as the ratchet permits movement in a first direction of the first end relative to the second end, changing a circumference of the loop configuration.

In certain embodiments, the elongate band further comprises a pawl spring that comprises the shape-memory material. In certain embodiments, the ratchet permits movement in a second direction of the first end relative to the second end, changing the circumference of the loop. In certain embodiments, the ratchet comprises a pawl and a detent. In certain embodiments, the assembly being configured to be formed as a loop within or around a portion of the stomach or esophagus, and wherein the elongate band is configured to change a dimension of a lumen of the portion of the stomach or esophagus by adjusting the circumference of the loop. In certain embodiments, wherein the assembly is configured to decrease the circumference of the loop. In certain embodiments, the ratchet further comprises a plurality of detents, the elongate band comprises a pawl at the second end of the elongate band, and the elongate band is configured to decrease or increase the circumference of the loop by drawing a detent past the pawl when the shape-memory material is activated. In certain embodiments, the elongate band is configured to be implanted around a portion of the stomach to form a gastric pouch, and the elongate band is configured to change a size of a lumen in the gastric pouch by adjusting a circumference of the loop. In certain embodiments, the elongate band comprises a polymer. In certain embodiments, the shape-memory material comprises at least one of a metal, a metal alloy, a nickel titanium alloy, and a shape-memory polymer. In certain embodiments, the shape-memory material comprises at least one of Fe—C, Fe—Pd, Fe—Mn—Si, Co—Mn, Fe—Co—Ni—Ti, Ni—Mn—Ga, Ni₂MnGa, and Co—Ni—Al. In certain embodiments, the elongate band is configured to detach into a first band portion and a second band portion. In certain embodiments, the adjustable implant further comprises a hydrophilic material substantially coating at least a portion of the implant. In certain embodiments, the hydrophilic material comprises at least one of polyethylene glycol and Poly 2-Hydroxyethylmethacrylate. In certain embodiments, an external layer having a varying thickness comprises the hydrophilic material. In certain embodiments, the adjustable implant further comprises a restraint that at least partially encloses at least a portion of the ratchet and is configured to prevent or reduce at least one of (1) the first end of the elongate band from uncoupling from the ratchet, and (2) encroachment by tissue into a region at the first end. In certain embodiments, the adjustable implant further comprises a spring return mechanism coupled to the elongate band.

In certain embodiments, an adjustable implant configured to be implanted around an outer surface of a stomach or esophagus is disclosed. The implant comprises encircling means for at least partially surrounding the stomach or esophagus, the encircling means comprising a shape-memory material. The implant further comprises ratchet means for permitting movement in a first direction of a first end of the encircling means relative to a second end of the encircling means. Activation of the shape-memory material adjusts the encircling means from a first length to a second length as the ratchet means permits the movement of the first end relative to the second end, changing a circumference of the encircling means.

In certain embodiments, the adjustable implant further comprises an activation means configured to provide an activation energy to the shape-memory material.

In certain embodiments, a method, for treating obesity, is disclosed. The method comprises placing an adjustable implant according to certain embodiments within or around a patient's stomach or esophagus. The method further comprises applying an activation energy to the shape-memory material. The method further comprises transforming the shape-memory material from a first configuration to a second configuration, thereby changing the circumference of the loop configuration.

In certain embodiments, the ratchet comprises a plurality of serially arranged detents, and changing the circumference of the loop configuration comprises moving the first end of the elongate band from a first position, at one of the plurality of detents, to a second position, at another of the plurality of detents. In certain embodiments, the implant further comprises a spring configured to expand the circumference of the loop configuration to a maximum circumference. In certain embodiments, the implant further comprises a hydrophilic coating that substantially coats at least a portion of the implant. In certain embodiments, the implant comprises a pre-implantation shape and a post-implantation shape, and the method further comprises laparoscopically inserting the implant in the pre-implantation shape into the patient, so as to facilitate having the implant assume the post-implantation shape around the stomach or esophagus. In certain embodiments, the implant comprises a pre-implantation shape and a post-implantation shape, and the method further comprises endoscopically inserting the implant in the pre-implantation shape into the patient, so as to facilitate having the implant assume the post-implantation shape within the stomach or esophagus. In certain embodiments, the shape-memory material comprises at least one of a metal, a metal alloy, a nickel titanium alloy, and a shape-memory polymer. In certain embodiments, the shape-memory material comprises at least one of Fe—C, Fe—Pd, Fe—Mn—Si, Co—Mn, Fe—Co—Ni—Ti, Ni—Mn—Ga, Ni₂MnGa, and Co—Ni—Al. In certain embodiments, the activation energy comprises at least one of magnetic resonance imaging energy, high-intensity focused ultrasound energy, radio frequency energy, x-ray energy, microwave energy, light energy, electric field energy, magnetic field energy, inductive heating, and conductive heating.

For purposes of summarizing the invention, certain aspects, advantages, and novel features of the invention have been described herein. It is to be understood that not necessarily all such advantages may be achieved in accordance with any particular embodiment of the invention. Thus, the invention may be embodied or carried out in a manner that achieves or optimizes one advantage or group of advantages as taught herein without necessarily achieving other advantages as may be taught or suggested herein.

### BRIEF DESCRIPTION OF THE DRAWINGS

A general architecture that implements the various features of the invention will now be described with reference to the drawings. The drawings and the associated descriptions are provided to illustrate embodiments of the invention and not to limit the scope of the invention. Throughout the drawings, reference numbers are re-used to indicate correspondence between referenced elements.

FIG. 1 is a front elevational view of a stomach that has undergone a Gastric Banding procedure using the prior art LAP-BAND® Adjustable Gastric Banding System;

FIG. 2 is a front elevational view of a stomach that has undergone a Gastric Banding procedure using one embodiment of the present dynamically adjustable gastric implants;

FIG. 3 is a front elevational view of the stomach of FIG. 2 after the implant has been adjusted;

FIG. 4 is a front elevational view of a stomach that has undergone a Gastric Banding procedure using another embodiment of the present dynamically adjustable gastric implants;

FIG. 5 is a front perspective view of one embodiment of the present dynamically adjustable gastric implants;

FIG. 6 is a front perspective view of the implant of FIG. 5 after the implant has been adjusted;

FIG. 7 is a front perspective view of the implant of FIG. 5 after the implant has been further adjusted from the configuration of FIG. 6;

FIG. 8 is a top plan view of another embodiment of the present dynamically adjustable gastric implants, illustrating the implant in a pre-adjusted configuration;

FIG. 9 is a top plan view of the implant of FIG. 8, illustrating the implant in a post-adjusted configuration;

FIG. 10 is a top plan view of another embodiment of the present dynamically adjustable gastric implants;

FIG. 11 is a top plan view of another embodiment of the present dynamically adjustable gastric implants;

FIG. 12 is a top plan view of another embodiment of the present dynamically adjustable gastric implants;

FIG. 13 is a top plan view of another embodiment of the present dynamically adjustable gastric implants;

FIG. 14 is a top plan view of another embodiment of the present dynamically adjustable gastric implants;

FIG. 15 is a detail view of the portion of the implant of FIG. 14 indicated by the line 15-15;

FIG. 16 is a top plan view of another embodiment of the present dynamically adjustable gastric implants, illustrating the implant in a pre-adjusted configuration;

FIG. 17 is a top plan view of the implant of FIG. 16, illustrating the implant in a post-adjusted configuration;

FIG. 18 is a top plan view of the implant of FIGS. 16 and 17, illustrating the pre-adjusted and post-adjusted configurations superimposed upon one another;

FIG. 19 is a top plan view of another embodiment of the present dynamically adjustable gastric implants, illustrating the implant in a pre-adjusted configuration;

FIG. 20 is a top plan view of the implant of FIG. 19, illustrating the implant in a post-adjusted configuration;

FIG. 21 is a front elevational view of another embodiment of the present dynamically adjustable gastric implants and a stomach, illustrating a configuration of the implant and stomach after activation of the implant;

FIG. 22 is a front elevational view of another embodiment of the present dynamically adjustable gastric implants and a stomach, illustrating a configuration of the implant and stomach after activation of the implant;

FIG. 23 is a front elevational view of another embodiment of the present dynamically adjustable gastric implants and a stomach, illustrating a configuration of the implant and stomach after activation of the implant;

FIG. 24 is a top plan view of another embodiment of the present dynamically adjustable gastric implants, illustrating the implant in a pre-adjusted configuration;

FIG. 25 is a top plan view of the implant of FIG. 24, illustrating the implant in a post-adjusted configuration;

FIG. 26 is a front perspective view of another embodiment of the present dynamically adjustable gastric implants;

FIG. 27 is a front elevational view of another embodiment of the present dynamically adjustable gastric implants;

FIG. 28 is a front elevational view of another embodiment of the present dynamically adjustable gastric implants, illustrating several different sizes of the embodiment;

FIG. 29 is a front perspective view of another embodiment of the present dynamically adjustable gastric implants;

FIG. 30 is a front elevational view of a stomach and esophagus, illustrating schematically one possible configuration for implantation of any of the implants of FIGS. 26-29;

FIG. 31 is a detail view of a portion of another embodiment of the present dynamically adjustable gastric implants;

FIG. 32 is a detail view of the portion of FIG. 31 after the implant has been adjusted;

FIG. 33 is a front elevational view of a patient and another embodiment of the present dynamically adjustable gastric implants, illustrating one method of adjusting the implant using direct application of electrical impulses;

FIG. 34 is a front elevational view of one step in a method of implanting any of the present implants using a balloon catheter;

FIG. 35A is a side view of an another embodiment of the present dynamically adjustable gastric implant;

FIG. 35B is an end view of the ratchet mechanism of FIG. 35A;

FIG. 36A illustrates a top view of one embodiment of a ratchet mechanism;

FIG. 36B illustrates a side view of the ratchet mechanism of FIG. 36A;

FIG. 37A illustrates a bottom view of a portion of the embodiment of FIG. 35A;

FIG. 37B illustrates a side view of a portion of the embodiment of FIG. 35A;

FIG. 38A illustrates a side view of an embodiment of the implant of FIG. 35A in its maximum diameter configuration;

FIG. 38B illustrates a side view of an embodiment of the implant of FIG. 35A in its minimum diameter configuration;

FIG. 39 illustrates a side view of the implant of FIG. 35A in its unstable minimum diameter return configuration, just prior to removing the actuation energy;

FIG. 40 illustrates a plot of internal stress versus temperature for a shape-memory material which can be used to power the device;

FIG. 41 illustrates an embodiment of the implant of FIG. 35A further comprising an external layer;

FIG. 42A illustrates an embodiment of the ratchet mechanism of the implant of FIG. 35A where the ratchet mechanism is in a single plane; and

FIG. 42B illustrates an embodiment of the implant of FIG. 35A further comprising a separable region.

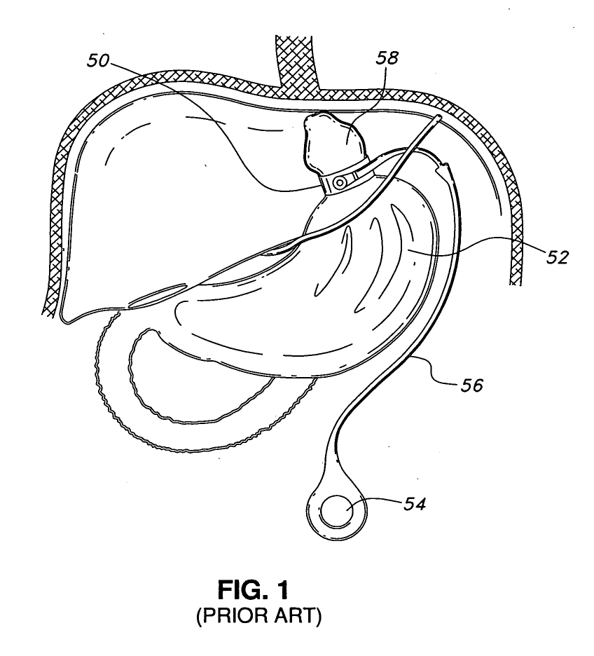

### DETAILED DESCRIPTION OF EXEMPLARY EMBODIMENTS

The present invention includes gastric implants and methods for dynamically restricting the capacity of a patient's stomach to treat obesity. As used herein, the term “gastric implant” describes an implant or implants that are configured for implantation within or around the outside of the stomach. Such implants are further configured to be dynamically adjusted, for example, by externally or internally activating the implant(s) to induce a change in shape and/or size of the implant(s).

In certain embodiments, an adjustable implant is implanted into the body of a patient such as a human or other animal. The adjustable implant may be disposed around the stomach, or within the stomach. The adjustable implant may also be disposed around the esophagus, or within the esophagus. The implant may be selected from one or more shapes comprising a ring shape (note that as used herein the term “ring” comprises both circular and non-circular shapes, and both open and closed configurations), an oval shape, a C-shape, a D-shape, a U-shape, an S-shape, a helical or coil shape, a cage shape, a wire stent shape and other shapes. The implant may be implanted through an incision during a traditional open procedure, such as a laparotomy, or endoscopically, or laparoscopically, or percutaneously, or through another type of procedure, as those of skill in the art will appreciate.

A variety of different implant locations are described below, including entirely within or around the stomach, and at the junction of the esophagus and the stomach. Those of skill in the art will appreciate that the present implants may be implanted anywhere within or around the stomach and/or the esophagus, and that multiple implants can be placed at different locations within the stomach and/or the esophagus. Further, the implants described herein can also be used in combination with other surgical procedures, such as Gastric Bypass, VBG, Duodenal Switch, etc.

The size and/or configuration of the present implants can be adjusted post-implantation through one of many techniques, including minimally invasive techniques and completely non-invasive techniques. For example, minimally invasive techniques include endoscopic, laparoscopic, percutaneous, etc. Completely non-invasive techniques include magnetic resonance imaging (MRI), application of high-intensity focused ultrasound (HIFU), inductive heating, a combination of these methods, etc. The implant may be adjusted at a time shortly after implantation in order to constrict and/or expand a portion of the stomach. The implant may also be adjusted at a later time in order to further constrict and/or expand the stomach and/or to allow a previously constricted portion of the stomach to expand and/or to allow a previously expanded portion of the stomach to constrict. As used herein, “post-implantation” refers to a time after implanting the implant and closing the body opening through which the implant was introduced into the patient's body.

In certain embodiments, the implant comprises a shape-memory material that is responsive to changes in temperature and/or exposure to a magnetic field. Shape-memory is the ability of a material to regain its shape after deformation. Shape-memory materials include polymers, metals, metal alloys and ferromagnetic alloys. The implant may be adjusted in vivo by applying an energy source to activate the shape-memory material and cause it to change to a memorized shape. As used herein, “activation” of a shape-memory material refers at least to the phenomenon of the shape-memory material undergoing a shape change in response to application of energy from an energy source. The energy source may include, for example, radio frequency (RF) energy, x-ray energy, microwave energy, ultrasonic energy such as focused ultrasound, HIFU energy, light energy, electric field energy, magnetic field energy, cryogenics, combinations of the foregoing, or the like. For example, one embodiment of electromagnetic radiation that is useful is infrared energy having a wavelength in a range between approximately 750 nanometers and approximately 1600 nanometers. This type of infrared radiation may be produced efficiently by a solid state diode laser. In certain embodiments, the implant may be selectively heated using short pulses of energy having an on and off period between each cycle. The energy pulses provide segmental heating, which allows segmental adjustment of portions of the implant without adjusting the entire implant.

In certain embodiments, the implant may include an energy-transmitting material to increase heating efficiency and localize heating in the area of the shape-memory material. Thus, damage to the surrounding tissue can be reduced or eliminated. Energy-transmitting materials for light or laser activation energy may include nanoshells, nanospheres and the like, particularly where infrared laser energy is used to energize the material. Such nanoparticles may be made from a dielectric, such as silica, coated with an ultra thin layer of a conductor, such as gold, and be selectively tuned to absorb a particular frequency of electromagnetic radiation. In certain such embodiments, the nanoparticles range in size between about 5 nanometers and about 20 nanometers and can be suspended in a suitable material or solution, such as saline solution. Coatings comprising nanotubes or nanoparticles can also be used to absorb energy from, for example, HIFU, MRI, inductive heating, or the like. In the case of MRI, the coating might include a specific resonance frequency other than the 64 MHz that is typically used in MRI. Thus, the implant can be imaged and controllably adjusted in size and/or shape by using two or more different frequencies of energy simultaneously. A tunable frequency can be used to better direct activation energy without impacting the image quality.

In certain embodiments, thin film deposition or other coating techniques such as sputtering, reactive sputtering, metal ion implantation, physical vapor deposition, and chemical deposition can be used to cover portions or all of the implant. Such coatings can be either solid or microporous. When HIFU energy is used, for example, a microporous structure may trap and direct the HIFU energy toward the shape-memory material. The coating improves thermal conduction and heat removal. In certain embodiments, the coating also enhances radio-opacity of the implant. Coating materials can be selected from various groups of biocompatible organic or non-organic, metallic or non-metallic materials such as titanium nitride (TiN), iridium oxide (Irox), carbon, graphite, ceramic, platinum black, titanium carbide (TiC) and other materials used for pacemaker electrodes or implantable pacemaker leads. Other materials discussed herein or known in the art can also be used to absorb energy.

In addition, or in certain embodiments, fine conductive wires such as platinum coated copper, titanium, tantalum, stainless steel, gold, or the like, may be wrapped around the shape-memory material to allow focused and rapid heating of the shape-memory material while reducing undesired heating of surrounding tissues.

In certain embodiments, the energy source is applied surgically either during implantation or at a later time using an activation means. For example, the shape-memory material can be heated during implantation of the implant by touching the implant with a warm object. As another example, the energy source can be surgically applied after the implant has been implanted by inserting a catheter into the patient's body and applying the energy through the catheter. The catheter may be inserted percutaneously, or through a peroral transgastric procedure, for example. Various types of energy, such as ultrasound, microwave energy, RF energy, light energy or thermal energy (e.g., from a heating element using resistance heating), can be transferred to the shape-memory material through a catheter positioned on or near the shape-memory material. Alternatively, thermal energy can be provided to the shape-memory material by injecting a heated fluid through a catheter or circulating the heated fluid in a balloon through the catheter placed in close proximity to the shape-memory material. As another example, the shape-memory material can be coated with a photodynamic absorbing material that is activated to heat the shape-memory material when illuminated by light from a laser diode or directed to the coating through fiber optic elements in a catheter. In certain such embodiments, the photodynamic absorbing material includes one or more drugs that are released when illuminated by the laser light.

In certain embodiments, a removable subcutaneous electrode or coil couples energy from a dedicated activation unit. In certain such embodiments, the removable subcutaneous electrode provides telemetry and power transmission between the system and the implant. The subcutaneous removable electrode allows more efficient coupling of energy to the implant with minimum or reduced power loss. In certain embodiments, the subcutaneous energy is delivered via inductive coupling.

In certain embodiments, the energy source is applied in a non-invasive manner from outside the patient's body. In certain such embodiments, the external energy source may be focused to provide directional heating to the shape-memory material so as to reduce or minimize damage to the surrounding tissue. For example, in certain embodiments, a handheld or portable device comprising an electrically conductive coil generates an electromagnetic field that non-invasively penetrates the patient's body and induces a current in the implant. The current heats the implant and causes the shape-memory material to transform to a memorized shape. In certain such embodiments, the implant may also comprise an electrically conductive coil wrapped around or embedded in the shape-memory material. The externally generated electromagnetic field induces a current in the implant's coil, causing it to heat and transfer thermal energy to the shape-memory material.

In certain embodiments, an external HIFU transducer focuses ultrasound energy onto the implant to heat the shape-memory material. In certain such embodiments, the external HIFU transducer is a handheld or portable device. The terms “HIFU,” “high intensity focused ultrasound” or “focused ultrasound” as used herein are broad terms and are used at least in their ordinary sense and include, without limitation, acoustic energy within a wide range of intensities and/or frequencies. For example, HIFU includes acoustic energy focused in a region, or focal zone, having an intensity and/or frequency that is considerably less than what is currently used for ablation in medical procedures. Thus, in certain such embodiments, the focused ultrasound is not destructive to the patient's organ tissue. In certain embodiments, HIFU includes acoustic energy within a frequency range of approximately 0.5 MHz and approximately 30 MHz and a power density within a range of approximately 1 W/cm² and approximately 500 W/cm².

In certain embodiments, the implant comprises an ultrasound absorbing material or hydro-gel material that allows focused and rapid heating when exposed to the ultrasound energy and transfers thermal energy to the shape-memory material. In certain embodiments, a HIFU probe is used with an adaptive lens to compensate for movement within the body due to, for example, respiration. The adaptive lens has multiple focal point adjustments. In certain embodiments, a HIFU probe with adaptive capabilities comprises a phased array or linear configuration. In certain embodiments, an external HIFU probe comprises a lens configured to be placed between a patient's ribs to improve acoustic window penetration and reduce or minimize issues and challenges regarding passing through bones.

In certain embodiments, HIFU or other activation energy can be synchronized with an imaging device, such as MRI, ultrasound or X-ray, to allow visualization of the implant during HIFU activation. The imaging device may include an algorithm to display the area of interest for energy delivery. In addition, or in certain embodiments, ultrasound imaging can be used to non-invasively monitor the temperature of tissue surrounding the implant by using principles of speed of sound shift and changes to tissue thermal expansion.

In certain embodiments, non-invasive energy is applied to the implant post-implantation using a Magnetic Resonance Imaging (MRI) device. In certain such embodiments, the shape-memory material is activated by a constant magnetic field generated by the MRI device. In addition, or in certain embodiments, the MRI device generates RF pulses that induce current in the implant and heat the shape-memory material. The implant can include one or more coils and/or MRI energy-transmitting material to increase the efficiency and directionality of the heating. Suitable energy-transmitting materials for magnetic activation energy include particulates of ferromagnetic material. Suitable energy-transmitting materials for RF energy include ferrite materials as well as other materials configured to absorb RF energy at resonant frequencies thereof.

In certain embodiments, the MRI device is used to determine the size of the implanted implant before, during and/or after the shape-memory material is activated. In certain such embodiments, the MRI device generates RF pulses at a first frequency to heat the shape-memory material and at a second frequency to image the implant. Thus, the size of the implant can be measured without heating the implant. In certain such embodiments, an MRI energy-transmitting material heats sufficiently to activate the shape-memory material when exposed to the first frequency and does not substantially heat when exposed to the second frequency. Other imaging techniques known in the art can also be used to determine the size of the implant including, for example, ultrasound imaging, computed tomography (CT) scanning, X-ray imaging, or the like. In certain embodiments, such imaging techniques also provide sufficient energy to activate the shape-memory material.

As discussed above, shape-memory materials include, for example, polymers, metals, and metal alloys including ferromagnetic alloys. Examples of shape-memory polymers that are usable for certain embodiments of the present implant are disclosed by Langer, et al. in U.S. Pat. No. 6,720,402, issued Apr. 13, 2004, U.S. Pat. No. 6,388,043, issued May 14, 2002, and U.S. Pat. No. 6,160,084, issued Dec. 12, 2000, each of which are hereby incorporated by reference herein. Shape-memory polymers respond to changes in temperature by changing to one or more permanent or memorized shapes. In certain embodiments, the shape-memory polymer may be heated to a temperature between approximately 38 degrees Celsius and approximately 60 degrees Celsius. In certain embodiments, the shape-memory polymer may be heated to a temperature in a range between approximately 40 degrees Celsius and approximately 55 degrees Celsius. In certain embodiments, the shape-memory polymer has a two-way shape-memory effect wherein the shape-memory polymer can be heated to change it to a first memorized shape and cooled to change it to a second memorized shape. The shape-memory polymer can be cooled, for example, by inserting or circulating a cooled fluid through a catheter.

Shape-memory polymers implanted in a patient's body can be heated non-invasively using, for example, external light energy sources such as infrared, near-infrared, ultraviolet, microwave and/or visible light sources. The light energy may be selected to increase absorption by the shape-memory polymer and reduce absorption by the surrounding tissue. Thus, damage to the tissue surrounding the shape-memory polymer is reduced when the shape-memory polymer is heated to change its shape. In certain embodiments, the shape-memory polymer comprises gas bubbles or bubble containing liquids such as fluorocarbons and is heated by inducing a cavitation effect in the gas/liquid when exposed to HIFU energy. In certain embodiments, the shape-memory polymer may be heated using electromagnetic fields and may be coated with a material that absorbs electromagnetic fields.

Certain metal alloys have shape-memory qualities and respond to changes in temperature and/or exposure to magnetic fields. Examples of shape-memory alloys that respond to changes in temperature include titanium-nickel, copper-zinc-aluminum, copper-aluminum-nickel, iron-manganese-silicon, iron-nickel-aluminum, gold-cadmium, combinations of the foregoing, and the like. In certain embodiments, the shape-memory alloy comprises a biocompatible material such as a titanium-nickel alloy.

Shape-memory alloys exist in two distinct solid phases called martensite and austenite. The martensite phase is relatively soft and easily deformed, whereas the austenite phase is relatively stronger and less easily deformed. For example, shape-memory alloys enter the austenite phase at a relatively high temperature and the martensite phase at a relatively low temperature. Shape-memory alloys begin transforming to the martensite phase at a start temperature (Mₛ) and finish transforming to the martensite phase at a finish temperature (M_{f}). Similarly, such shape-memory alloys begin transforming to the austenite phase at a start temperature (Aₛ) and finish transforming to the austenite phase at a finish temperature (A_{f}). Both transformations have a hysteresis. Thus, the Mₛ temperature and the A_{f} temperature are not coincident with each other, and the M_{f} temperature and the Aₛ temperature are not coincident with each other.

In certain embodiments, the shape-memory alloy is processed to form a memorized shape in the austenite phase in the form of a ring or partial ring. The shape-memory alloy is then cooled below the M_{f} temperature to enter the martensite phase and deformed into a larger or smaller ring. In certain such embodiments, the shape-memory alloy is sufficiently malleable in the martensite phase to allow a user such as a physician to adjust the circumference of the ring in the martensite phase by hand to achieve a desired fit for a particular stomach. After the ring is attached to the stomach, the circumference of the ring can be adjusted non-invasively by heating the shape-memory alloy to an activation temperature (e.g., temperatures ranging from the Aₛ temperature to the A_{f} temperature).

Thereafter, when the shape-memory alloy is exposed to a temperature elevation and transformed to the austenite phase, the alloy changes in shape from the deformed shape to the memorized shape. Activation temperatures at which the shape-memory alloy causes the shape of the implant to change shape can be selected and built into the implant such that collateral damage is reduced or eliminated in tissue adjacent the implant during the activation process. Examples of A_{f} temperatures for suitable shape-memory alloys range between approximately 45 degrees Celsius and approximately 70 degrees Celsius. Furthermore, examples of Mₛ temperatures range between approximately 10 degrees Celsius and approximately 20 degrees Celsius, and examples of M_{f} temperatures range between approximately −1 degrees Celsius and approximately 15 degrees Celsius. The size of the implant can be changed all at once or incrementally in small steps at different times in order to achieve the adjustment necessary to produce the desired clinical result.

Certain shape-memory alloys may further include a rhombohedral phase, having a rhombohedral start temperature (Rₛ) and a rhombohedral finish temperature (R_{f}), that exists between the austenite and martensite phases. An example of such a shape-memory alloy is a NiTi alloy, which is commercially available from Memry Corporation (Bethel, Conn.). In certain embodiments, an example of an Rₛ temperature range is between approximately 30 degrees Celsius and approximately 50 degrees Celsius, and an example of an R_{f} temperature range is between approximately 20 degrees Celsius and approximately 35 degrees Celsius. One benefit of using a shape-memory material having a rhombohedral phase is that in the rhombohedral phase the shape-memory material may experience a partial physical distortion, as compared to the generally rigid structure of the austenite phase and the generally deformable structure of the martensite phase.

Certain shape-memory alloys exhibit a ferromagnetic shape-memory effect wherein the shape-memory alloy transforms from the martensite phase to the austenite phase when exposed to an external magnetic field. The term “ferromagnetic” as used herein is a broad term and is used in its ordinary sense and includes, without limitation, any material that easily magnetizes, such as a material having atoms that orient their electron spins to conform to an external magnetic field. Ferromagnetic materials include permanent magnets, which can be magnetized through a variety of modes, and materials, such as metals, that are attracted to permanent magnets. Ferromagnetic materials also include electromagnetic materials that are capable of being activated by an electromagnetic transmitter, such as one located outside the stomach. Furthermore, ferromagnetic materials may include one or more polymer-bonded magnets, wherein magnetic particles are bound within a polymer matrix, such as a biocompatible polymer. The magnetic materials can comprise isotropic and/or anisotropic materials, such as for example NdFeB (neodymium-iron-boron), SmCo (samarium-cobalt), ferrite and/or AlNiCo (aluminum-nickel-cobalt) particles.

Thus, an implant comprising a ferromagnetic shape-memory alloy can be implanted in a first configuration having a first shape and later changed to a second configuration having a second (e.g., memorized) shape without heating the shape-memory material above the Aₛ temperature. Advantageously, nearby healthy tissue is not exposed to high temperatures that could damage the tissue. Further, since the ferromagnetic shape-memory alloy does not need to be heated, the size of the implant can be adjusted more quickly and more uniformly than by heat activation.

Examples of ferromagnetic shape-memory alloys include Fe—C, Fe—Pd, Fe—Mn—Si, Co—Mn, Fe—Co—Ni—Ti, Ni—Mn—Ga, Ni₂MnGa, Co—Ni—Al, and the like. Certain of these shape-memory materials may also change shape in response to changes in temperature. Thus, the shape of such materials can be adjusted by exposure to a magnetic field, by changing the temperature of the material, or both.

In certain embodiments, combinations of different shape-memory materials are used. For example, implants according to certain embodiments comprise a combination of shape-memory polymer and shape-memory alloy (e.g., NiTi). In certain such embodiments, an implant comprises a shape-memory polymer tube and a shape-memory alloy (e.g., NiTi) disposed within the tube. Such embodiments are flexible and allow the size and shape of the implant to be further reduced without impacting fatigue properties. In addition, or in certain embodiments, shape-memory polymers are used with shape-memory alloys to create a bi-directional (e.g., capable of expanding and contracting) implant. Bi-directional implants can be created with a wide variety of shape-memory material combinations having different characteristics.

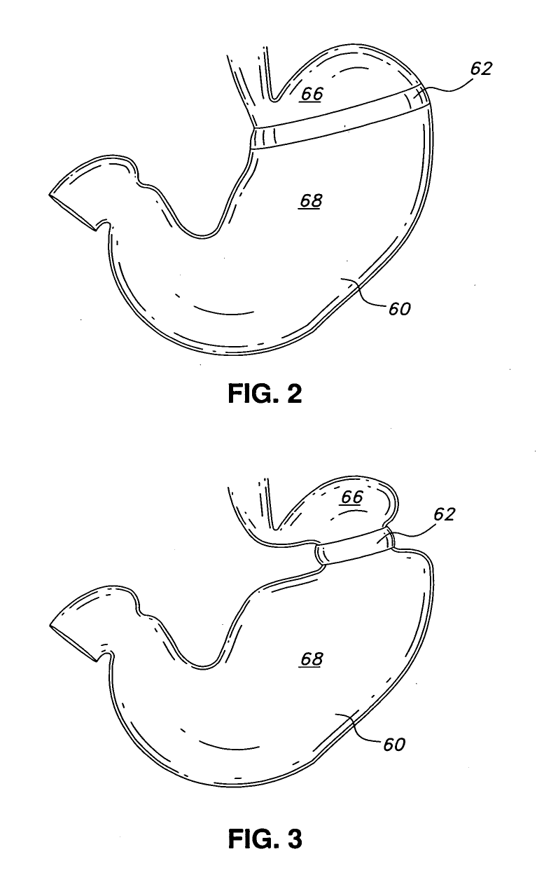

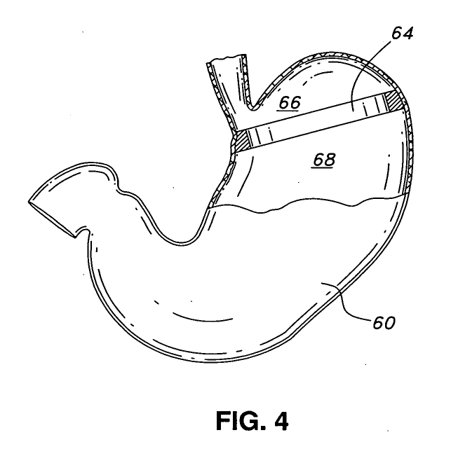

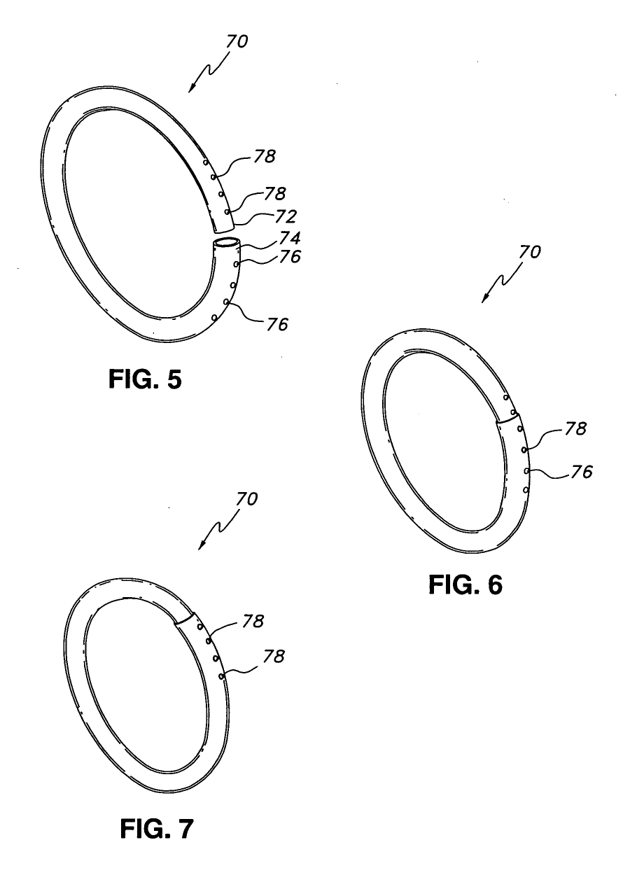

The present embodiments provide a system, method, and various devices to dynamically remodel and resize the stomach as the patient's needs change. For example, FIGS. 2 and 3 illustrate the pre- and post-adjustment configurations of a stomach 60 and one embodiment of a generally ring-shaped implant 62. In FIGS. 2 and 3 the implant 62 is configured to be disposed around the exterior surfaces of the stomach 60. FIG. 4 illustrates the pre-adjustment configuration of a stomach 60 and another embodiment of a generally ring-shaped implant 64 that is configured to be disposed within the stomach 60. The size and shape of each implant 62, 64 can be selected based upon the patient's anatomy. FIGS. 5-29, discussed in detail below, illustrate some examples of possible shapes.

FIGS. 2 and 4 illustrate the implants immediately after implantation, prior to any adjustments in the size and/or shape of the implants. In the illustrated configuration each of the generally ring-shaped implants forms a dividing line that separates the stomach into two regions. An upper region 66 includes the fundus, at least a portion of the cardia, and a portion of the body. A lower region 68 includes a portion of the body and the pylorus. Those of ordinary skill in the art will appreciate that the implants may be positioned and oriented in any of a variety of different ways from that illustrated. The exact positioning and orientation of the implants can be determined by the implanting physician according to the patient's needs.

The position of the implant relative to the stomach can be secured in any of a variety of ways. For example, sutures, staples, tacks, pins, and/or adhesives may secure the implant to the stomach. Stapling methods may include automatic or manual stapling. Adhesives may include, for example, tissue glue, heat activated glue, UV-curable glue, and room temperature or moisture activated glue. Securing and/or suturing of the various implant embodiments to the tissue can include a variety of energy sources, such as RF heating, laser, microwave, ultrasound, etc. Securing and/or suturing of the various implant embodiments to the tissue can be done all around the implant perimeter or at one or more points or segments. In certain embodiments, the implant may include one or more holes or suture rings through which sutures may pass, as described in more detail below.

FIG. 3 illustrates the stomach 60 and the external implant 62 of FIG. 2 after adjustments have been made to the size of the implant. As in FIG. 2, the generally ring-shaped implant separates the stomach into an upper region 66 and a lower region 68. The upper region forms a gastric pouch that can only hold a small amount of food. A stoma (not shown) connects the upper and lower regions. As the size of the implant decreases from the configuration of FIG. 2 to that of FIG. 3, the size of the stoma shrinks, thus limiting the rate at which food can pass from the upper stomach pouch to the lower region. Depending upon the patient's needs, the physician can activate the implant to achieve a smaller size, and thus a smaller stoma, from that illustrated in FIG. 3. Alternatively, during the activation procedure(s) the physician can stop short of the size illustrated in FIG. 3 so that the implant is configured to have a larger size, and thus a larger stoma, from that illustrated. As those of skill in the art will appreciate, the stomach and the internal implant 64 of FIG. 4 can be manipulated in a fashion similar to that just described for the external implant of FIGS. 2 and 3.

In certain embodiments the shape-memory material of the implant may be bi-directional, so that it is capable of expanding and contracting. With such an embodiment, the physician can dynamically adjust the size and/or shape of the implant as the patient's needs change. For example, a patient may have a need to lose a large amount of weight quickly. In such a case it may be advantageous to shrink the implant down to a relatively small size soon after implantation. The relatively small implant would then create a relatively small stoma so that the speed at which the patient could digest food would be greatly diminished, and the patient would lose weight relatively quickly. As the patient loses weight, his or her needs may change, and the physician may need to expand the implant to create a larger stoma, and thereby increase the speed at which the patient can digest food. With a bi-directional implant, the physician could easily expand the implant using one or more of the non-invasive techniques described above.

FIGS. 5-7 illustrate one embodiment of a generally ring-shaped implant 70 that may be used in the methods described above and illustrated in FIGS. 2-4. The implant 70 comprises a ring with a male end 72 that telescopically engages a female end 74. FIGS. 5-7 represent a possible time-lapse transformation of the implant 70 from a deformed shape (FIG. 5) to a memorized shape (FIG. 7). As an activating energy (such as heat, or a magnetic field, or any of the other energies described above) is applied to the implant of FIG. 5, the circumference of the implant becomes progressively smaller as the implant returns to its memorized shape, shown in FIG. 7. As the implant becomes progressively smaller, it cinches the portion of the stomach around which it is wrapped, decreasing the size of the stoma that connects the upper gastric pouch to the lower stomach region. In order to achieve a desired circumference for the implant after it has been implanted, and thus achieve a desired circumference for the stoma, the physician may halt the application of activation energy before the implant returns to its memorized shape. For example, the application of activation energy may be halted when the implant occupies the intermediate configuration of FIG. 6.

In the illustrated embodiment, the implant 70 includes retaining features that help the implant to maintain its shape after the application of activation energy has ceased. The female end 74 includes a plurality of evenly spaced holes 76. The male end 72 includes at least one protrusion 78. As activation energy is applied to the implant 70, and it contracts from the configuration of FIG. 5 toward the configuration of FIG. 7, the at least one protrusion 78 advances from one hole 76 to the next along the female end 74 as the male end 72 advances into the female end. Engagement of the at least one protrusion with each hole resists any tendency of the male end to withdraw from the female end. These retaining features thus help the implant 70 to remain in its contracted state even as the contracted stomach and/or esophagus apply pressure against the implant that might otherwise cause the implant to expand toward the configuration of FIG. 5. If the implant includes a plurality of protrusions 78 and holes 76, as illustrated, then an increasing number of protrusions and holes will engage one another as the male end advances into the female end. As the number of engaged features increases, so does the retaining power of the implant.

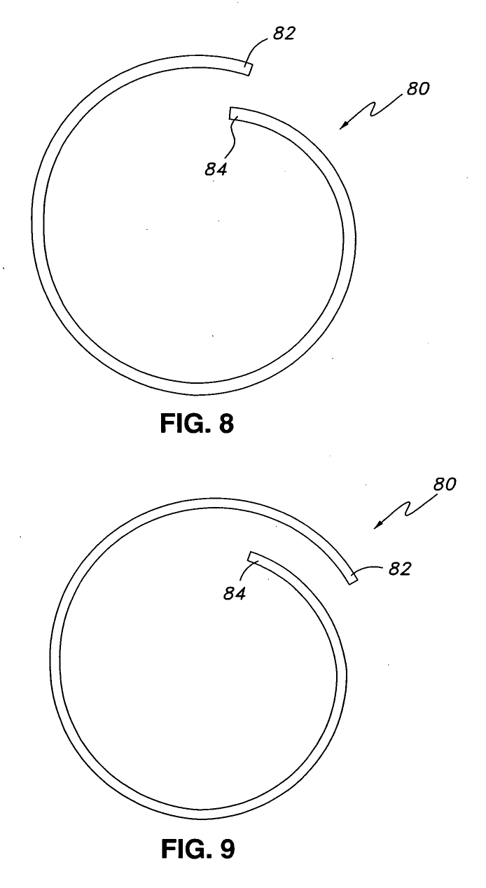

Those of ordinary skill in the art will appreciate that the implant 70 shown in FIGS. 5-7 is representative of a family of implants having a generally ring-shaped configuration. A variety of implants having a generally ring-shaped configuration could be produced to meet the needs of a wide variety of patients. For example, a generally ring-shaped implant may include ends that do not telescope or even overlap. FIGS. 8 and 9 illustrate another embodiment of a generally ring-shaped implant 80. The implant 80 resembles the implant shown in FIGS. 5-7, and includes first and second ends 82, 84 that overlap, but are not in contact with one another. FIG. 8 illustrates a pre-activation configuration, while FIG. 9 illustrates a post-activation configuration. As the implant 80 transforms from the pre-activation configuration (FIG. 8) to the post-activation configuration (FIG. 9), an amount of overlap of the ends 82, 84 increases as a circumference of the implant tightens.

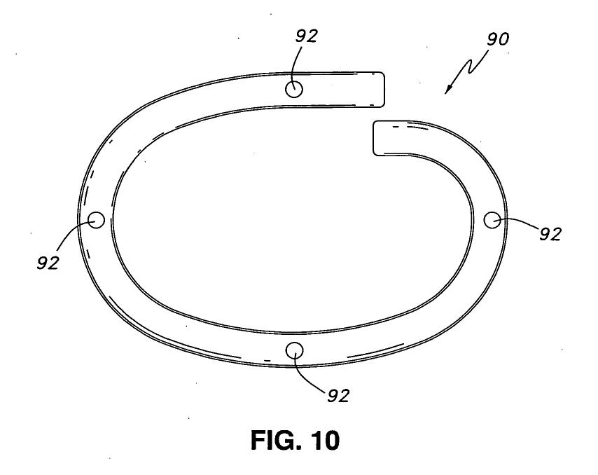

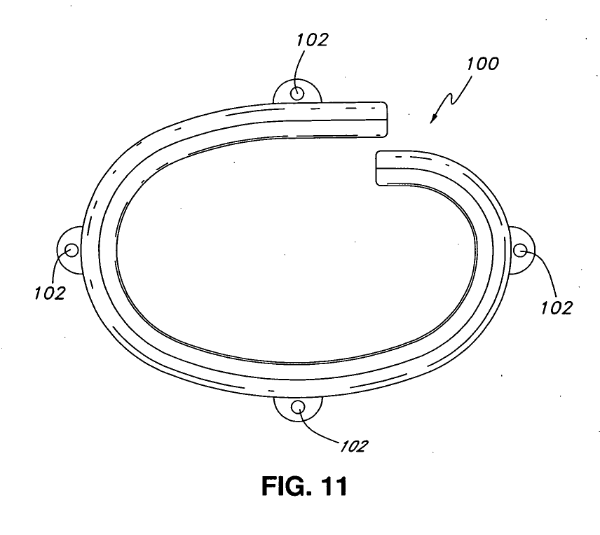

All of the embodiments of implants described herein may include features that facilitate the securing of the implant to the stomach and/or esophagus. For example, FIGS. 10-12 illustrate further embodiments of an implant 90, 100, 110 that is shaped substantially as an oval ring with overlapping ends. The implant 90 of FIG. 10 includes four evenly spaced suture holes 92, and the implant 100 of FIG. 11 includes four evenly spaced suture rings 102. In the illustrated embodiments, a longitudinal axis of each suture hole/ring extends in a direction substantially perpendicular to a plane defined by the implant. However, those of skill in the art will appreciate that the holes/rings could be oriented differently with respect to the implant. Each hole/ring may receive one or more sutures that may be used to secure the implant to the stomach. Those of ordinary skill in the art will appreciate that fewer or more suture holes/rings may be provided, and that they need not be evenly spaced. Those of ordinary skill in the art will also appreciate that suture holes/rings may be used with any of the implants described herein, and with implants of any shape or size.

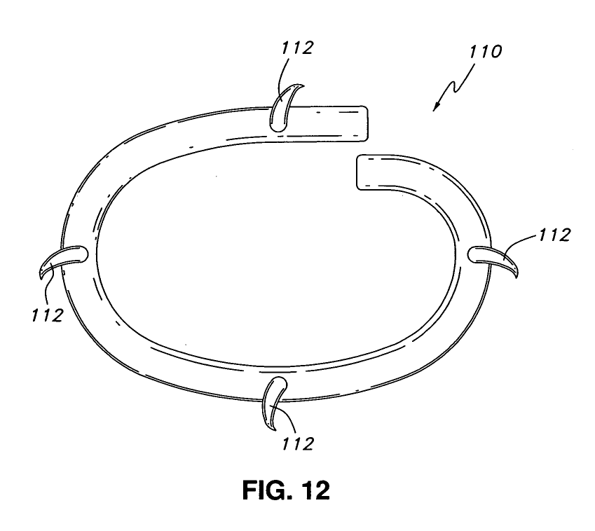

The implant 110 of FIG. 12 includes four evenly spaced hooks or barbs 112. Each hook or barb includes a sharp point that is adapted to penetrate and grip tissue. The hooks or barbs thus secure the implant 110 to the stomach. Those of ordinary skill in the art will appreciate that fewer or more hooks or barbs may be provided, and that they need not be evenly spaced. Those of ordinary skill in the art will also appreciate that hooks or barbs may be used with any of the implants described herein, and with implants of any shape or size.

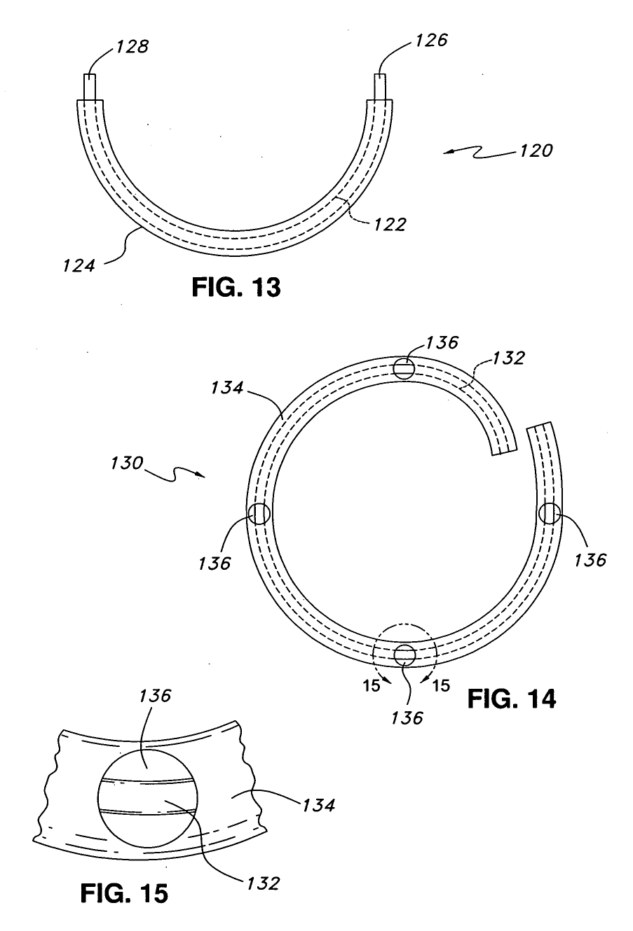

All of the embodiments of implants described herein may also include a cover. For example, FIG. 13 illustrates another embodiment of an implant 120 that is shaped substantially as a half ring, and FIG. 14 illustrates another embodiment of an implant 130 that is shaped substantially as a coiled ring with overlapping ends. Each implant 120, 130 includes a core 122, 132 formed of a shape-memory material and a cover 124, 134 disposed over the core. The cover 124, 134 may be constructed of any biodegradable and/or biocompatible material, such as polytetrafluoroethylene (PTFE) and expanded polytetrafluoroethylene (ePTFE). The cover may include multiple layers, such as an insulating layer and a polymer jacket. The cover may serve as a protective barrier between the core and any surrounding tissue, and may help the implant to become integrated into the surrounding tissue. For example, the cover 124, 134 may be constructed of a porous material or a fabric. Such porous materials or fabrics can be impregnated with a time-release substance, such as anti-inflammatory drugs, anti-obesity drugs, a combination thereof, or other drugs. The cover may also comprise a lubricious coating, such as polylactic acid (PLA), that eases placement and/or removal of the implant. The cover may also comprise a anti-inflammatory coating to minimize inflammation response. The cover may also aid in suturing the implant to the tissue by acting as a medium that sutures can penetrate. A surgeon implanting one of the present implant embodiments may pass a suturing needle first through the cover and then through the tissue to secure the implant to the tissue.

Depending upon the composition of the cover, it may insulate the core so that the core is less readily able to absorb activating energy and undergo a shape change. Accordingly, in the embodiment 120 of FIG. 13 at a first end and a second end of the implant the core 122 extends beyond the cover 124 to form a first exposed core portion 126 and a second exposed core portion 128. Similarly, in the embodiment of FIG. 14, the cover 134 includes four evenly spaced openings 136 that expose short lengths of the core 132. FIG. 15 illustrates a detail view of one of the openings 136 and the core 132. The exposed portions of the core may create locations where the core is readily able to absorb activating energy, which can then be conducted along the core to the non-exposed portions. The exposed portions thus provide locations at which activation energy can be focused, which both reduces energy loss during activation and reduces the likelihood that surrounding tissue might absorb unfocused activation energy and become damaged through overheating. In addition, any tissue in contact with an insulated portion of the implant is protected from absorbing heat through conduction from the implant.

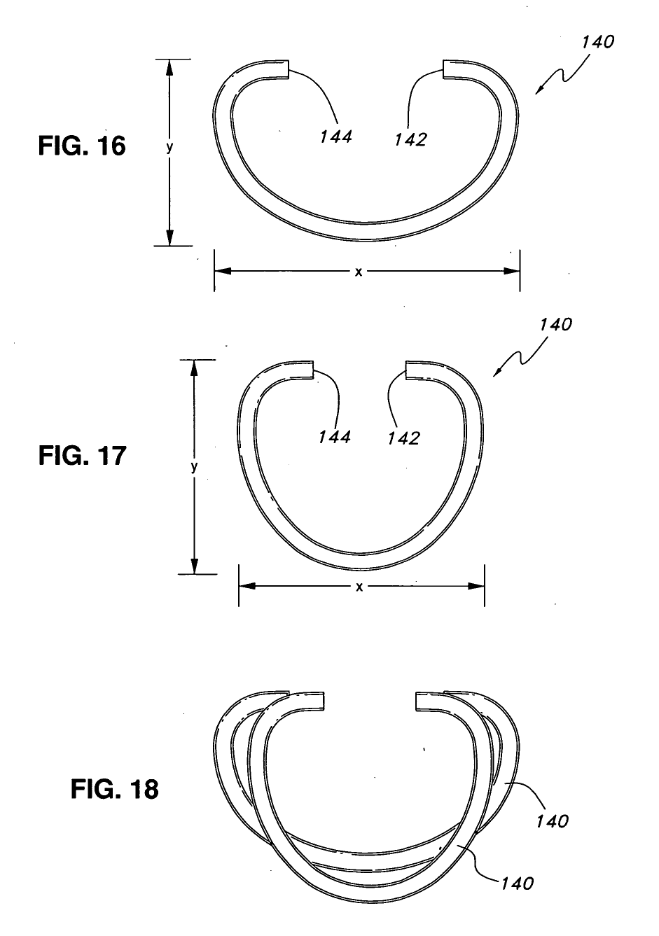

FIGS. 16 and 17 illustrate another embodiment of a generally ring-shaped implant 140. The implant resembles the letter C, and includes first and second ends 142, 144 that do not overlap one another. FIG. 16 illustrates a pre-activation configuration for the implant 140, while FIG. 17 illustrates a post-activation configuration. As with all of the implant embodiments described herein, the implant may be implanted either within the stomach and/or esophagus, or around the outside of the stomach and/or esophagus. In one embodiment of a method of implantation, the implant may be implanted in the pre-activation configuration, and then activated to induce a shape change. The activation may take the form of any of the methods described above, or any equivalent method.

In the pre-activation configuration, the implant includes a width dimension x and a height dimension y. As FIG. 18 illustrates, in the post-activation configuration the width dimension x of the implant is decreased, while the height dimension y of the implant is increased. Thus, no matter where the implant is placed on or in the stomach and/or esophagus, it reshapes and resizes the stomach and/or the esophagus to alter a path of travel of food through these areas, and/or to alter a patient's ability to absorb nutrients.

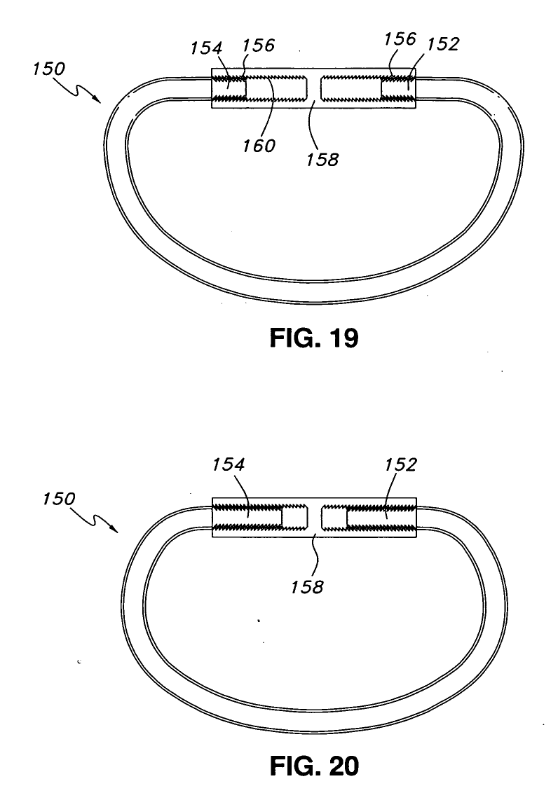

FIGS. 19 and 20 illustrate another embodiment of a generally ring-shaped implant 150. The implant 150 is similar in shape to the implant 140 shown in FIGS. 16 and 17, and includes first and second ends 152, 154 that do not overlap. FIG. 19 illustrates a pre-activation configuration, while FIG. 20 illustrates a post-activation configuration. Each of the implant ends 152, 154 includes ratchet teeth 156. A ratchet sleeve 158 receives each of the ends 152, 154. The sleeve 158 includes ratchet teeth 160 that are complementary to the teeth 156 on the implant ends. Thus, as the implant 150 progresses from the pre-activation configuration to the post-activation configuration the implant ends 152, 154 advance into the sleeve 158, and the mating ratchet teeth 156, 160 resist any tendency of the ends 152, 154 to withdraw from the sleeve 158. Because the implant ends are held firmly in the sleeve, there is less likelihood that the implant might relax and cause an unwanted change in shape of the stomach and/or esophagus.

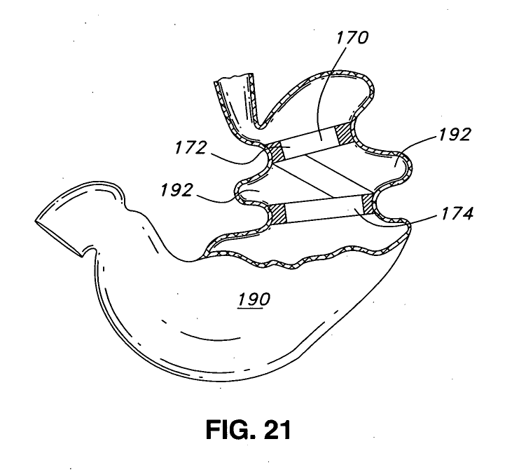

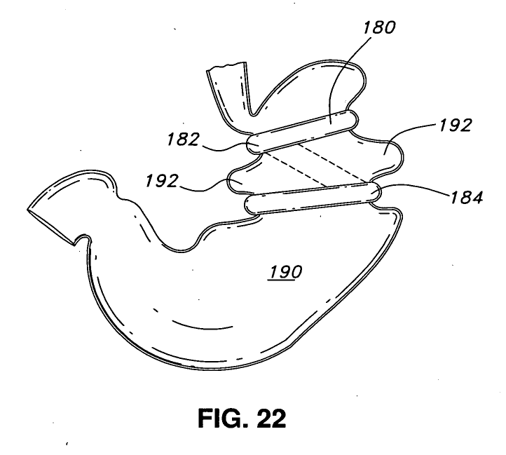

FIGS. 21 and 22 illustrate additional embodiments of the present implants 170, 180. Each of the implants 170, 180 comprises a generally helical shape with approximately two turns. As those of skill in the art will appreciate, a generally helical implant could have any number of turns.

In the illustrated embodiments, each implant 170, 180 is secured to and constricts an upper portion of the stomach 190. In FIG. 21 the implant is 170 disposed within the stomach, while in FIG. 22 the implant 180 is disposed around the outside of the stomach. As with all of the implant embodiments described herein, the implants 170, 180 of FIGS. 21 and 22 could be secured to the stomach 190 using any of the methods described herein, such as suturing, stapling, adhesives, etc., or any equivalent methods. Further, and again as with all of the implant embodiments described herein, the implants of FIGS. 21 and 22 could include apparatus to facilitate the securing of the implants, such as suture holes/rings, hooks, anchors, etc., and could include a cover.

FIGS. 21 and 22 illustrate the implants 170, 180 in a post-activation configuration. The upper turn 172, 182 and lower turn 174, 184 of each helical implant squeeze the stomach 190, constricting an upper portion of the stomach and creating a relatively narrow channel through which food can pass. The relatively narrow channel slows the passage of food, slowing the patient's digestion and making the patient feel full more quickly. The helical shape of the implants 170, 180 also shortens in length upon activation, creating bulges 192 in the stomach in the areas of the stomach that are located between adjacent turns. This deformation of the stomach creates a longer, tortuous path within the stomach for food to travel as it is being digested. The tortuous food path further reduces food intake, leading to additional weight loss benefits.

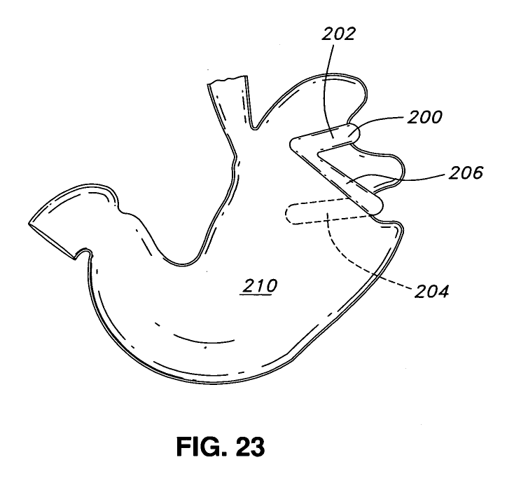

FIG. 23 illustrates another embodiment of the present implants. The implant 200 is shaped substantially as a Z, including an upper curved segment 3602, a lower curved segment 204 and an intermediate segment 206 joining the upper and lower segments. The intermediate segment 204 may be substantially straight, or it may be curved. The implant 200 is adapted to be disposed on one side of the stomach 210, either on the outside as illustrated, or on the inside. In the illustrated embodiment, the implant is disposed at the upper portion of the stomach, spanning the border between the fundus and the body. Those of skill in the art will appreciate, however, that the implant could be positioned anywhere on the stomach and/or esophagus. FIG. 23 illustrates the implant 200 in a post-activation configuration. Like the helical embodiments described above, the implant is adapted to constrict the stomach/esophagus to narrow the food passageway and alter a path of travel of food through the stomach/esophagus.

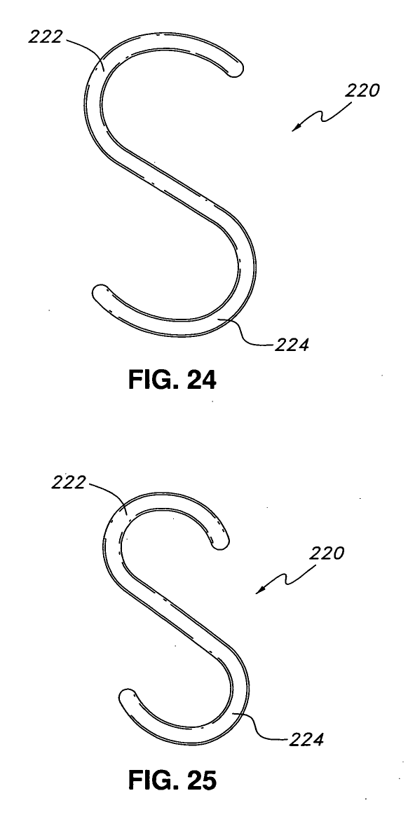

FIGS. 24 and 25 illustrate another embodiment of the present implants having a substantially S-shaped configuration. The implant 220 is adapted to be secured to one side of the stomach/esophagus, either within the stomach/esophagus or around the outside thereof. For example, the implant 220 could be positioned at the upper portion of the stomach, spanning the border between the fundus and the body. FIG. 24 illustrates the implant 220 in a pre-activation configuration, while FIG. 25 illustrates the implant in a post-activation configuration. As the implant transitions from the configuration of FIG. 24 to that of FIG. 25, an upper coil 222 and a lower coil 224 of the S tighten, thereby constricting tissue in two different places and forming upper and lower bulges in the stomach/esophagus. As with previous embodiments, the tightening of the implant constricts the stomach/esophagus to narrow the food passageway and alter a path of travel of food through the stomach/esophagus.

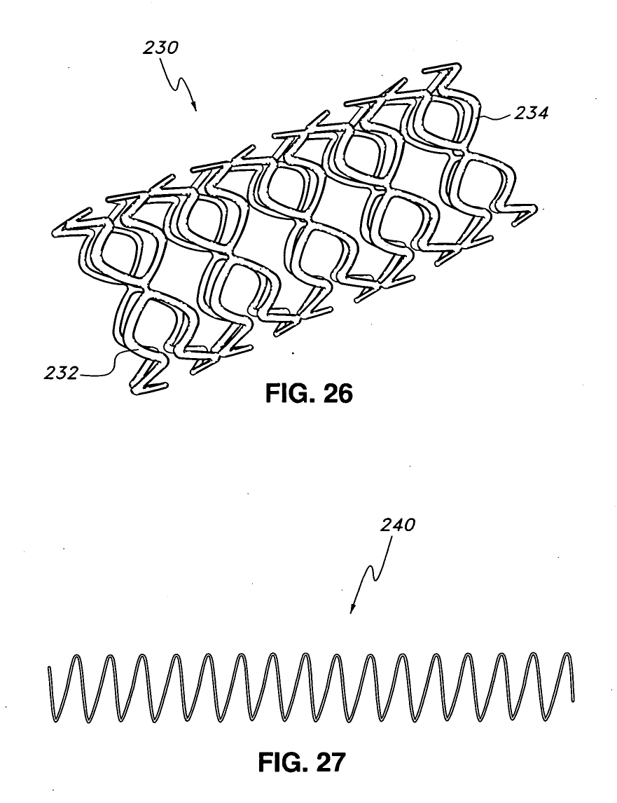

FIGS. 26-29 illustrate alternative implant configurations. These implants 230, 240, 250, 260 are modeled after typical vascular stents. For example, the implants 230, 250, 260 of FIGS. 26, 28 and 29 each resemble a tubular stent, while the implant 240 of FIG. 27 resembles a coil stent. The implants 230, 250, 260 of FIGS. 26, 28 and 29 each comprise a plurality of interconnected wire-like members that form a tubular cage structure. Those of ordinary skill in the art will appreciate that the illustrated configurations of the interconnected members are merely examples, and that implants having alternate configurations are fully equivalent to the illustrated implants.

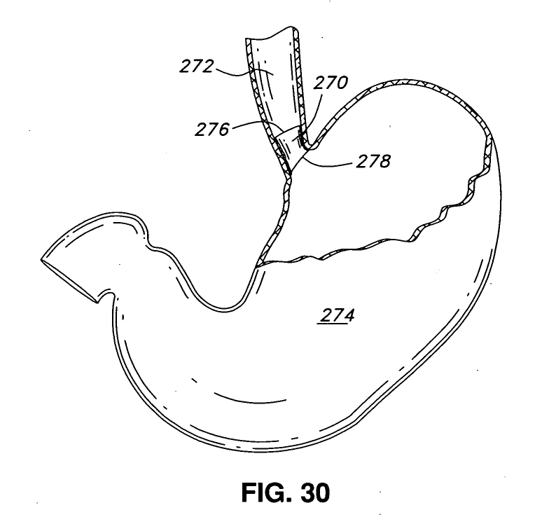

FIG. 30 illustrates, schematically, one possible configuration for implanting any of the implants of FIGS. 26-29. FIG. 30 shows a schematic configuration of an implant 270, the esophagus 272 and the stomach 274 shortly after implantation, and before any activation energy has been applied to the implant 270. In the illustrated embodiment, the implant 270 is located at the junction of the esophagus 272 and the stomach 274. An upper end 276 of the implant is located below the esophageal sphincter, while a lower end 278 of the implant extends into the stomach. Either end of the implant may be secured to the organ tissue, while portions of the implant in between the ends may also be secured to the tissue. While the illustrated implant is located within the esophagus and the stomach, those of skill in the art will appreciate that the implant could be located around the outside of these organs. Those of skill in the art will appreciate that any of the implants disclosed herein could also be located at the junction of the esophagus and the stomach. Those of skill in the art will also appreciate that the implants of FIGS. 26-29 could be implanted entirely within the stomach, or around the outside of the stomach.

When activation energy is applied to the implant 270 shown in FIG. 30, it may contract, thereby constricting the stomach/esophagus to narrow the food passageway and alter a path of travel of food through the stomach/esophagus. The extent of organ tissue constricted depends upon how much of the implant is secured to the stomach/esophagus.

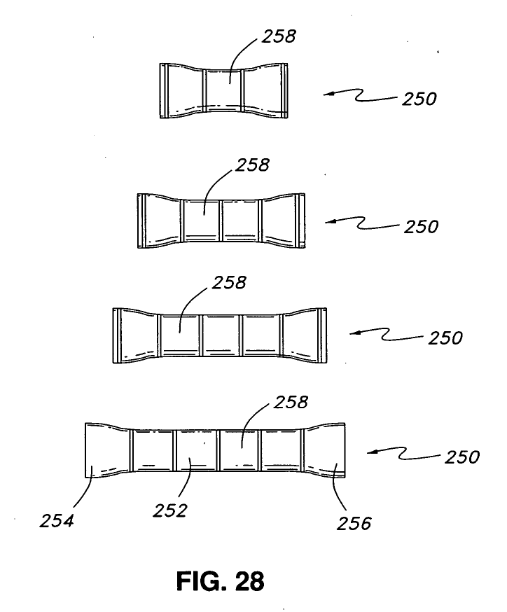

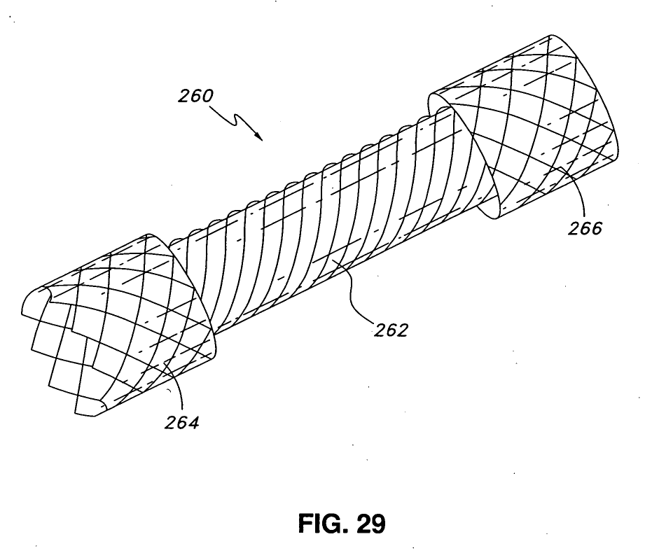

In FIG. 26, the implant 230 has a constant diameter from a first end 232 to a second end 234. In FIG. 28, the implant 250 has a constant diameter along an intermediate segment 252, then flares outwardly to a larger diameter at either end 254, 256. In FIG. 29, the implant 260 has a constant diameter along an intermediate segment 262, then abruptly transitions to a larger diameter at either end 264, 266. With the implants 250, 260 of FIGS. 28 and 29, the transition from the large opening at the proximal end 254, 264 to the relatively small intermediate section 252, 262 allows the implants to bring food slowly into the stomach, since the food will slow down at the bottleneck. Food will also exit the implant more quickly through the relatively wide distal end 256, 266.

Possible dimensions for the generally tubular implants of FIGS. 26-29 include the following. If the implant is to be positioned at the junction of the esophagus and the stomach, the implant might be between 5 mm and 50 mm in diameter, and between 20 and 200 mm in length. If the implant is to be positioned within or around the outside of the stomach, the implant might be between 20 mm and 100 mm in diameter, and between 20 and 200 mm in length.

In the embodiment 250 of FIG. 28, several different lengths of the implant are shown, and the cage-like structure of the implant is concealed by a sleeve 258. The sleeve 258 is analogous to the cover discussed above with respect to the embodiments having a shape-memory core and a cover. The sleeve 258 may thus be constructed of any of the materials discussed above with respect to the cover, and share any of the same properties discussed above with respect to the cover.

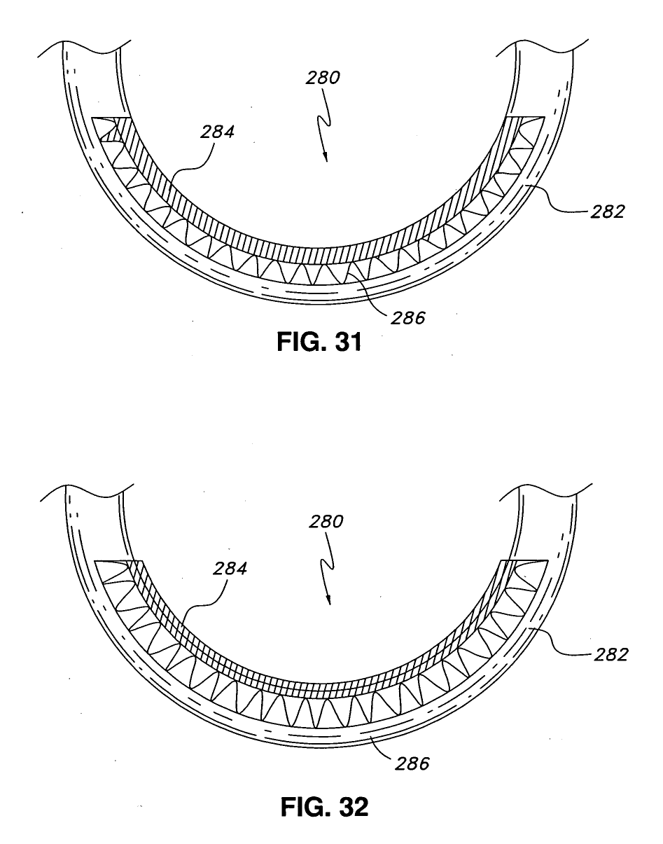

FIGS. 31 and 32 illustrate one possible configuration for any of the implants disclosed herein. The implant segment 280 includes a frame 282 constructed of a material that does not have a shape-memory. For example, the frame 282 could be constructed of a metal or a polymer. Along an interior surface (a surface that will contact the stomach/esophagus) the frame 282 includes band 284 of a flexible material. For example, the band 284 could be constructed of silicone rubber. Disposed just behind the band is a layer of a shape-memory material 286. In the illustrated embodiment, the shape-memory material has a coiled configuration. However, those of skill in the art will appreciate that the shape-memory material layer could have any configuration.

FIG. 31 illustrates the implant segment 280 in a pre-adjusted configuration, while FIG. 32 illustrates the implant segment 280 in a post-adjusted configuration. In FIG. 31 the inner band 284 is substantially flush with the inner surface of the frame 282. After the shape-memory material 286 is activated, the inner band 284 is pushed outward away from the inner surface and into the configuration shown in FIG. 32. If an implant having the configuration of FIGS. 31 and 32 is disposed around the outside of a stomach/esophagus, the inner band 284 will constrict the stomach/esophagus as it is pushed away from the inner surface.

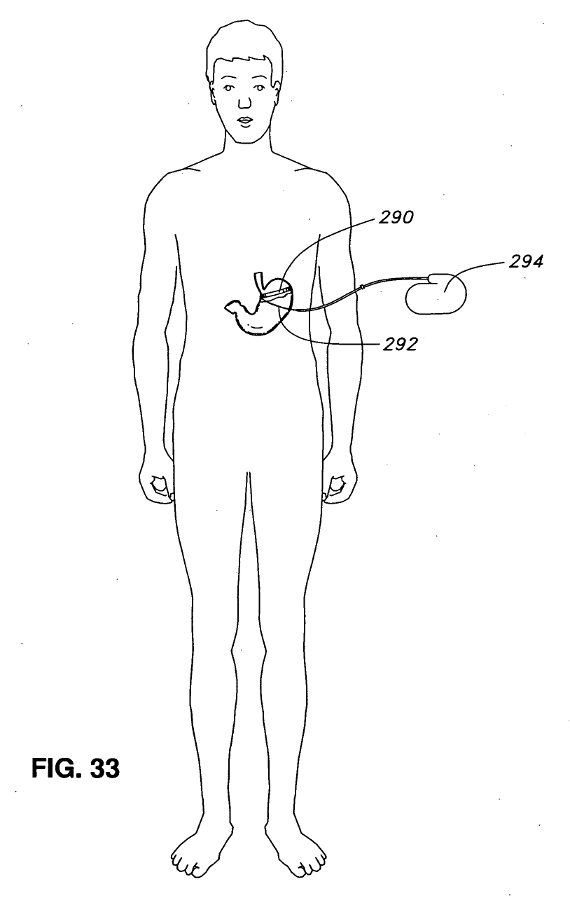

As discussed above, the size and/or configuration of any of the present implants may be adjusted post-implantation through one of many techniques, including minimally invasive techniques (endoscopic, laparoscopic, percutaneous, etc.) and completely non-invasive techniques (MRI, HIFU, inductive heating, a combination of these methods, etc.). FIG. 33 illustrates one example of a minimally invasive technique. The implant 290 may be directly connected to an electrical lead 292 that passes through the patient's skin. An external end of the lead may be connected to an electronic device 294 that is configured to generate electrical impulses. The lead 292 may transmit the impulses to the implant 290, generating activation energy within the implant in the form of heat.

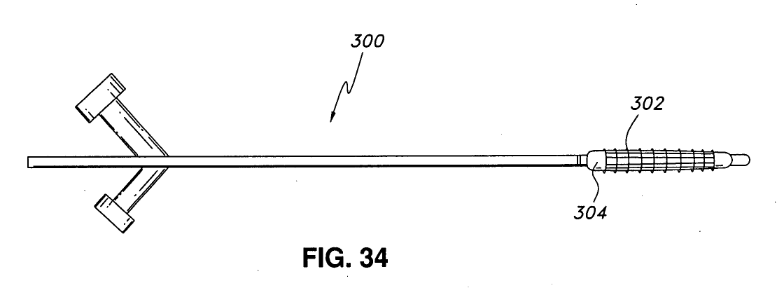

Also as discussed above, the present implants may be implanted in any of a variety of ways, such as during a traditional open procedure, or endoscopically, or laparoscopically, or percutaneously, or through another type of procedure. FIG. 34 illustrates one method of implanting the present implants using a balloon catheter 300. The implant 302 may be loaded over the balloon 304, and the balloon advanced to the implantation site. Once the implant reaches the implantation site, the balloon may be inflated to expand the implant. After the balloon is deflated and removed from the implantation site, the expanded implant can be secured to the stomach/esophagus using any of the methods described above. While FIG. 34 illustrates a generally tubular implant, those of skill in the art will appreciate that the balloon catheter implantation method can be used with any of the implants described herein.

Two-Way Adjustable Implant

In certain embodiments, a bi-directionally adjustable gastric implant (“bi-directional gastric implant”) is disclosed. Certain embodiments of the bi-directional gastric implant may be used according to the methods described above, such as in conjunction with vertical banded gastroplasty. In certain embodiments, the bi-directional gastric implant comprises a hollow ring-like structure with two ends. In certain embodiments, the ring-like structure of the implant is greater in diameter than the axial length of the implant. In certain embodiments, the bi-directional gastric implant has a central opening. In certain embodiments, a bi-directional gastric implant may comprise an adjustable band. In certain embodiments, the band comprises a plurality of detents (or teeth) along one surface. The band may be configured to expand the gastric implant by successively engaging the detents. After a final detent is reached, the implant may then return to its original, contracted position and may then repeat the expansion process anew.

Certain embodiments of the bi-directional gastric implant disclose a substantially circular band or sleeve that comprises a shape-memory element, a return spring element, a ratchet mechanism further comprising a plurality of detents, a pawl mechanism, a return mechanism, and an optional capsule for motion control and tissue isolation. These elements, when assembled according to certain embodiments disclosed herein, may comprise an actuator capable of a plurality of cycles, wherein during each cycle the band may constrict diametrically and expand diametrically due to circumferential length changes. The ratchet and pawl mechanism may be protected an optional restraint (or capsule) to permit undisturbed movement of the active material, and prevent encroachment by tissue or surgical procedures.

In certain embodiments, implantation of the bi-direction gastric implant around the stomach may facilitate the creation of a gastric pouch on the proximal to the ring. As discussed herein, with respect to the gastrointestinal tract, a proximal side of the implant refers to the side of the implant that is closer to the mouth, and a distal side of the implant refers to the side of the implant that is closer to the anus. Thus, the movement of food during normal digestion occurs in an antegrade direction, from the proximal end of the distal end.

In certain embodiments, the bi-directional gastric implant device may actuate in a linear to effect a simple length change from shorter to longer, or longer to shorter. In yet another embodiment, the device can comprise a single arcuate shape that subtends less than a 360 degree circle. In these embodiments, a first layer slides across a second layer forcing the first layer to undergo bending.

In certain embodiments, the ratchet elements may be moved in a forward direction but may be inhibited from moving in a reverse direction because the ratchet detents catch on the pawl when forced in the reverse direction. In the forward direction, the pawl slides over a ramp to avoid catching on the ratchet detents. Additional forward movement of the pawl over the ratchet detents, generated by increasingly higher amounts of activation energy applied to the shape-memory element, permit locking at discreet positions determined by the location where the forward edge of each ratchet detent engages the backward facing edge of the pawl. The ratchet elements engage to permit locking of the device in a given configuration once actuation energy is removed.

In certain embodiments, application of sufficient activation energy to cause the shape-memory elements to become heated to a temperature in excess of the A_{f} temperature may cause the shape-memory elements to force the pawl past the last ratchet detent. At this point, the pawl disengages from the ratchet detents. In certain embodiments, when the energy source is removed, and/or when the shape-memory elements cool to a temperature below M_{f}, the return spring pulls the pawl back to the beginning of the ratchet set where it is ready for another cycle of controlled advancement. In certain embodiments, the pawl is disengaged from the ratchet detents by bending upward. In certain embodiments, the pawl is disengaged from the ratchet detents by configuring the last ratchet detent with an angle that moves the pawl to the side into a return track. In certain embodiments, the pawl is disengaged from the ratchet detents by tilting the rearward facing surface of the pawl so that it cannot engage the forward edge of the ratchet detents. In certain embodiments, the pawl reconfiguration can be powered by mechanical energy as when the pawl hits the forward most end of the ratchet. In certain embodiments, the pawl can be tilted or disengaged by a shape-memory response at a specific temperature.

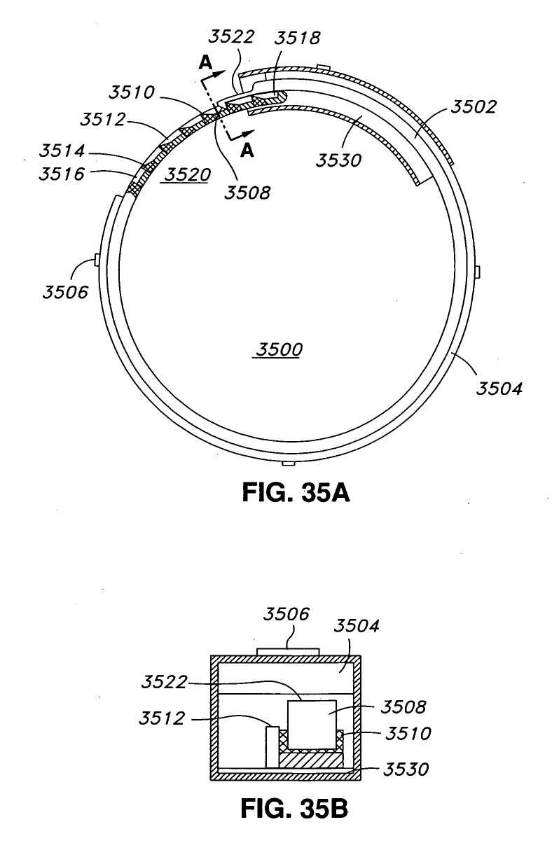

FIG. 35A illustrates a side view of a bi-directional gastric implant 3500 comprising a main support 3502, a return spring 3504, a plurality of band attachments 3506, and a ratchet assembly 3520 further comprising a plurality of detents 3510, a ratchet end wall 3512, a return detent 3514, a return guide 3516, a start guide channel 3518, a pawl 3508, and a pawl spring 3522. FIG. 35B illustrates an end view of the ratchet mechanism of FIG. 35A. In certain embodiments, the adjustable band implant 3500 may further comprise an optional restraint (or capsule) 3530.

In certain embodiments, the bi-directional gastric implant 3500 is a composite structure with the return spring 3504 affixed to the main support 3502 in a plurality of locations defined by the band attachments 3506. In certain embodiments, the pawl 3508 is affixed to, or is integrally formed with, the pawl spring 3522. The pawl spring 3522 may be affixed to, or integral to, the underside of the main support 3502 at a first end, while the upper side of the main support 3502 comprises, at a second end, a ratchet assembly. In certain embodiments, the detents 3510, the ratchet end wall 3512, the return detent 3514, the return guide 3516, and the start guide channel 3518, may be integrally formed within the main support 3502. The pawl 3508 may also be affixed to the main support 3502 at the end opposite that of the ratchet assembly 3520. In certain embodiments, the ratchet mechanism 3520 and the pawl 3508 may be affixed to other materials of the implant 3500. For example, the return spring 3504 may be configured to be affixed to the ratchet assembly 3520 or the pawl 3508. The restranint 3530 may be affixed to the ratchet mechanism 3520 and may be disposed to surround, at least in part, the end with the pawl 3508. In another embodiment, the restranint 3530 is affixed to the end with the pawl 3508, and surrounds, at least in part, the ratchet mechanism 3520. In certain embodiments, the free end, to which the restranint 3530 is not affixed, may be slidably disposed to move within the restraint or capsule 3530 with minimum friction.

In certain embodiments, the return spring 3502 may be affixed to the main support 3504 using a plurality of band attachments 3506. For example, the return spring 3502 may be affixed to the main support 3504 using rivets, clips, pins, bolts, or the like, inserted through holes or slots in the return spring 3504 and the main support 3502. In certain embodiments, the band attachment 3506 may be a slot within the main support 3502 into which a structure on the return spring 3504 is inserted to prevent disengagement of the return spring 3504 from the main support 3502. In certain embodiments, the band attachment slot may take any shape, such as a dovetail shape, with the insertion structure taking a complementary shape. In certain embodiments, in order to accommodate for variations in circumferential motion due to different radial positions, a composite construction as described can benefit from the use of circumferentially oriented elongated slots rather than holes in the return spring 3504 or the main support 3502 so that a small amount of compensatory translation can occur to maintain optimal alignment.

In certain embodiments, the pawl 3508 may be a rigid structure as illustrated in FIG. 35A. In certain embodiments, the pawl 3508 may be affixed to a spring, such as a leaf spring or a coil spring, to allow the pawl 3508 to deflect upward away from the ramp on the back side of each detent 3514. In certain embodiments, each detent 3514 may be configured with a ramp on one edge while the other edge is configured to be vertical or undercut in order to facilitate adjustment of the implant 3500. In certain embodiments, each detent 3514 may be configured to have a ramp on both edges. In certain embodiments, the detents 3514 may be depressions in a surface of the ratchet 3520, such as grooves. In certain embodiments, the detents 3514 may be protrusions from a surface of the ratchet 3520, as illustrated.

In certain embodiments, the ratchet 3520 may be manufactured from plastic and/or biocompatible polymers. The polymer can comprise, for example, polycarbonate, silicone rubber, polyurethane, silicone elastomer, a flexible or semi-rigid plastic, combinations of the same and the like. In certain embodiments, the ratchet 3520 may comprise one or more biocompatible materials known in the art, for example, polyester (Dacron®), polyamide (Nylon®, Delrin®), polyimide (PI), polyetherimide (PEI), polyetherketone (PEEK), polyamide-imide (PAI), polyphenylene sulfide (PPS), polysulfone (PSU), silicone, woven velour, polyurethane, polytetrafluoroethylene (PTFE, Teflon®), expanded PTFE (ePTFE), fluoroethylene propylene (FEP), perfluoralkoxy (PFA), ethylene-tetrafluoroethylene-copolymer (ETFE, Tefzel®), ethylene-chlorotrifluoroethylene (Halar®), polychlorotrifluoroethylene (PCTFE), polychlorotrifluoroethylene (PCTE, Aclar®, Clarus®), polyvinylfluoride (PVF), polyvinylidenefluoride (PVDF, Kynar®, Solef®), fluorinated polymers, polyethylene (PE, Spectra®), polypropylene (PP), ethylene propylene (EP), ethylene vinylacetate (EVA), polyalkenes, polyacrylates, polyvinylchloride (PVC), polyvinylidenechloride, polyether block amides (PEBAX), polyaramid (Kevlar®), heparin-coated fabric, or the like. In certain embodiments, the ratchet 3520 may be manufactured from metal, such as, but not limited to, titanium or stainless steel. In certain embodiments, the main support 3502, the return spring 3504, the plurality of band attachments 3506, the pawl 3508, and the pawl spring 3522 may be manufactured from plastic or biocompatible polymers, such as a nylon plastic. In certain embodiments, the main support 3502, the return spring 3504, the plurality of band attachments 3506, the pawl 3508, and the pawl spring 3522 may be manufactured from metal. In certain embodiments, substantially all materials of the implant, except for the main support 3502 and/or pawl spring 3522, may be manufactured from plastic and/or biocompatible polymers.

In certain embodiments, the main support 3502 comprises a shape-memory material, as described above. In certain embodiments, the pawl spring 3522 comprises a shape-memory material.

In certain embodiments, the implant 3500 may have a diameter of between about 5 mm and about 50 mm, and a length of between about 20 and about 200 mm. In certain embodiments, the implant 3500 may have a diameter of between about 20 mm and about 100 mm, and a length of between about 10 mm and about 400 mm. In certain embodiments, a cross-section of implant 3500 may have a width of between about 0.5 mm and about 4 mm. In certain embodiments, a cross-section of implant 3500 may have a width of between about 0.25 mm and about 6 mm. In certain embodiments, a cross-section of implant 3500 may have a height of between about 1 mm and about 10 mm. In certain embodiments, a cross-section of implant 3500 may have a height of between about 0.5 mm and about 10 mm.

In certain embodiments, the return spring 3504 may have a length of between about 0.25 mm and about 10 mm. In certain embodiments, the main support 3502 may have a length of between about 60 mm and about 300 mm. In certain embodiments, the optional restranint 350 may have a width of between about 0.5 mm and about 4 mm. In certain embodiments, the optional restranint 350 may have a height of between about 1 mm and about 10 mm. In certain embodiments, the optional restranint 350 may have a length of between about 5 mm and about 200 mm. In certain embodiments, a detent 3510 may have a height of between about 0.5 mm and about 5 mm. In certain embodiments, a detent 3510 may have a length of between about 0.5 mm and about 5 mm. In certain embodiments, the distance between consecutive detents may be between about 0.5 mm and about 10 mm. In certain embodiments, the pawl spring 3522 may have a length of between about 0.5 mm and about 10 mm. In certain embodiments, the return slot 3602 may have a length of between about 5 mm and about 100 mm.

In certain embodiments, the adjustable band implant 3500 may comprise an optional restranint 3530 surrounding at least a portion of the ratchet mechanism 3520. In certain embodiments, the restranint 3530 may surround at least a portion of the pawl 3508. The restranint 3530 may constrain the ratchet mechanism 3520 to move in a substantially longitudinal direction relative to the pawl. This restranint 3530 may be configured to keep the pawl 3508 from pulling away and becoming disengaged from the ratchet mechanism 3520. For example, the restranint 3530 may be configured like a stay on a belt. In certain embodiments, the restranint 3530 may comprise one or more biocompatible materials known in the art, as discussed above.

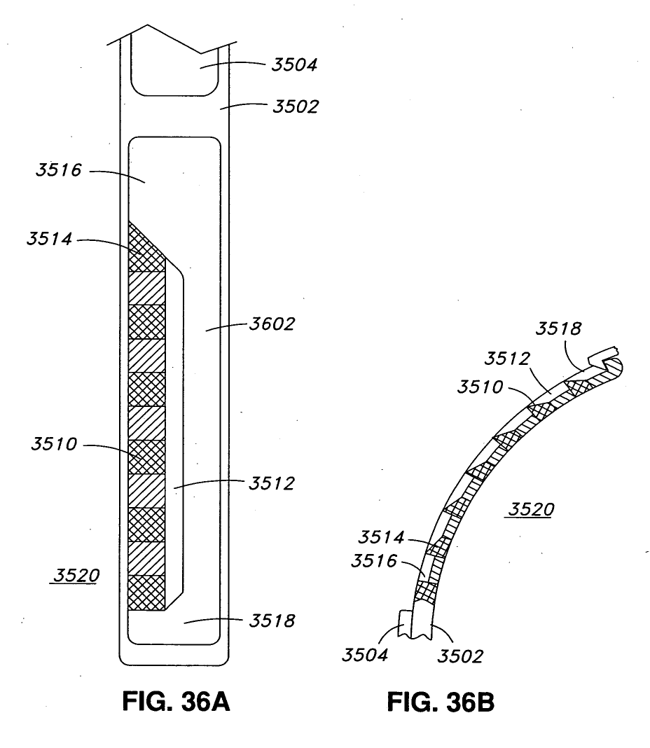

FIG. 36A illustrates a top view of one embodiment of a ratchet mechanism 3520. FIG. 36B illustrates a side view of the ratchet mechanism 3520 of FIG. 36A. In certain embodiments, the ratchet mechanism 3520 comprises a main support 3502, a return spring 3504, a plurality of detents 3510, a ratchet end wall 3512, a return detent 3514, a return guide 3516, a start guide channel 3518, and a return slot 3602.

In certain embodiments, the main support 3502, the plurality of detents 3510, the ratchet end wall 3512, the return detent 3514, the return guide 3516, the start guide channel 3518, and the return slot 3602 may be integrally formed into the main support 3502 using procedures such as electron discharge machining (EDM), photochemical etching, standard machining, or the like.

In certain embodiments, the detents 3510 generate gradually increasing force against forward motion of the engaged pawl 3508, as illustrated in FIG. 35. In certain embodiments, an implant has a maximum diameter in its start state, when the pawl 3508 is engaged to the start guide channel 3518. In certain embodiments, the implant may be adjusted (ex. contracted) by deflecting the pawl 3508 around the ramp. In certain embodiments, such adjustment relieves the force placed on the detents 3510 by the pawl 3508. Once the pawl 3508 has been moved past a detent 3510, it may fall into a low energy state due to recoil of the pawl spring 3522. The forward-facing wall of the detent 3510 may then engage the backward-facing wall of the pawl 3508 in order to prevent backward motion of the pawl 3508, thus creating a plurality of gates that may deter backward motion (or adjustment) of the pawl 3508, but may permit forward motion. In certain embodiments, both the forward-facing and the backward-facing walls are configured to pair with one another. For example, both walls may be substantially perpendicular, as illustrated.

In the illustrated embodiment, the pawl 3508 laterally moves (deflection) in a clockwise direction towards the left side of the ratchet end wall 3512 by passing over the detents 3510 during adjustment. In certain embodiments, the movement of the pawl is triggered by the activation of the shape-memory main support 3502 by the application of activation energy to that shape-memory main support 3502, as described above, such as by using a catheter.

In certain embodiments, activation energy may be delivered using a standalone pill. The pill may emit a signal detectable by an external receiver to allow an operator to track the progress of the pill as it passes through internal anatomy. While the pill is progressing through the open lumen, i.e., the area without the implantable band 3500, it may emit a tone detectable by a receiver. A visual indicator or display could also be provided to verify its position. When the pill enters the banded area, a tone or other indication from the pill and/or receiver may verify detection of the implant 3500 as well as confirm the pill is in position to communicate with and transfer energy to the implanted band 3500. With a suitable positional relationship established, the energy required to exercise or adjust the implant 3500 may be applied. Pressure on the underlying tissue may also be monitored by placing transducers at the tissue interface.

In certain embodiments, the pill configuration may consist of a pill sized module affixed to a flexible tether that could be swallowed by the patient. The position of the pill may be monitored by the external receiver, as described above, and adjusted through traction or relief on the tether. In certain embodiments, the tether may be restrained at its proximal end to control the position of the pill manually. In certain embodiments, the tether may be restrained at its proximal end to control the position of the pill through automated means. In certain embodiments, the pill may act as a sending and receiving station capable of receiving activation power from an external source and/or interrogate the device positional parameter, such as position, adjusted diameter position, and stage of adjustment. By combining this information with the parameters desired by the operator, activation energy may be transmitted to the pill and retransmitted to the implantable device 3500 with continuous telemetry monitoring to confirm initial and final diameter as well as temperature achieved by the implantable device 3500 and tissue temperature. In certain embodiments, a control unit may comprise safeguards to detect position changes, telemetry link breaks as well as the ability to interrupt power application if tissue temperature exceeds safe levels or device temperature inadequate for ratchet motor activation. In certain embodiments, a safety protocol may govern system operation.

In certain embodiments, the implant 3500 may be in a fully contracted state when the pawl 3508 is engaged to the detent immediately previous to the final (return) detent 3514. After the pawl 3508 has passed the return detent 3514, the pawl 3508 may enter the return guide 3516, wherein the removal of energy from the main support 3502 causes the return spring 3504 to dominate the force balance on the pawl 3508, thus causing the pawl 3508 to be pulled back against the return detent 3514. In order to facilitate entry of the pawl 3508 into the return guide 3516, in certain embodiments, the return detent 3514 has a substantially forward-angled surface which may cause the pawl 3508 to be deflected sideways into the return slot 3602. Upon entry to the return slot 3602, the pawl 3508 may be pulled to the beginning of the return slot 3602 where it returns to a starting position by entering the start guide channel 3518. The adjustment process may then be repeated from the start state.

The ratchet assembly 3520 and pawl 3508 may thus be continuously and repeatably adjusted. After each adjustment, activation energy may be discontinued so that the structure maintains a stable configuration in its un-energized state.

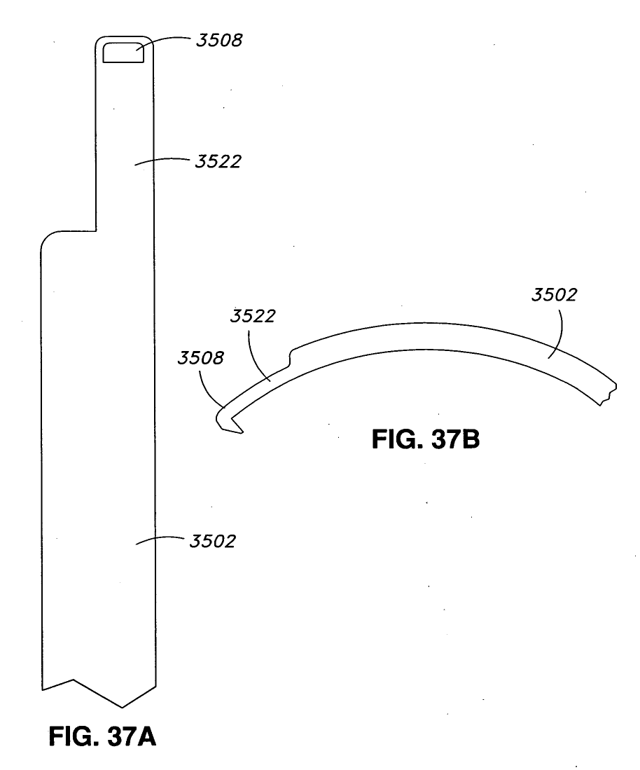

FIG. 37A illustrates a bottom view of the main support 3502 portion of the embodiment of FIG. 35A. FIG. 37B illustrates a side view of the main support 3502 portion of the embodiment of FIG. 35A. The illustrated portion of the main support 3502 includes the pawl 3508 and pawl spring 3522. In the illustrated embodiment, the pawl 3508 is affixed to the pawl spring 3522, which is affixed to the main support 3502. In certain embodiments, the pawl 3508 and/or pawl spring 3508 may be integrally formed with other portions of the implant 3500. The pawl 3508 is aligned to a neutral position so that it moves in its non-laterally deflected state with the ratchet mechanism 3520. In certain embodiments, the lateral motion of the pawl 3508 may occur due to movement of the entire end of the main support 3502. In certain embodiments, the lateral motion of the pawl 3508 may occur due to lateral deflection of the pawl spring 3522.

In certain embodiments, the pawl spring 3522 may be a leaf spring, as illustrated. In certain embodiments, a pawl spring 3522 may be fabricated from materials such as, but not limited to, Elgiloy®, superelastic nitinol, shape-memory nitinol, cobalt-nickel alloy, titanium, stainless steel, or other shape-memory materials, as described above. In certain embodiments, the pawl spring 3522 can comprise a coil spring. In certain embodiments, other types of springs may be used. In embodiments where the pawl spring 3522 comprises a shape-memory material, the pawl spring 3522 may be configured to disengage or move in a certain direction upon reaching a pre-determined activation temperature or state. In certain embodiments where the pawl spring 3522 comprises a shape-memory material, the pawl spring 3522 may be activated instead of, or in addition to, the main support 3502 in order to adjust the implant 3500.

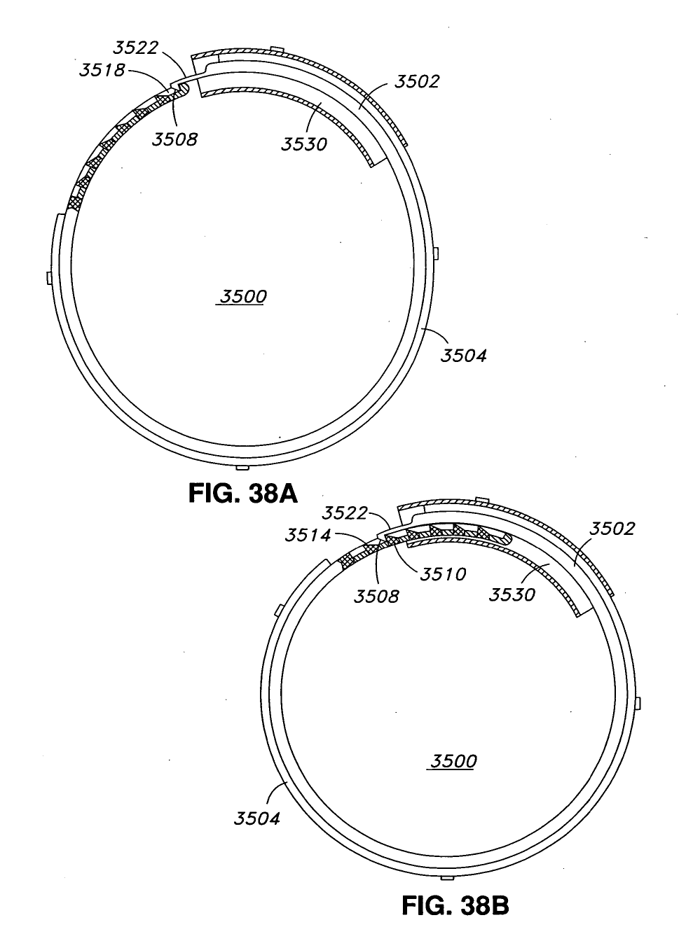

FIG. 38A illustrates a side view of an embodiment of the implant of FIG. 35A in its maximum diameter configuration. The implant 3500 comprises the sleeve 3530, the main support 3502, the return spring 3504, the pawl 3508, the pawl spring 3522, and the start guide channel 3518. In the maximum diameter configuration, the pawl 3508 has been drawn toward the start guide channel 3518 and rests therein. The pawl 3508 may be constrained to move in the forward direction at this point. Forward forces, such as those imparted by activation of the shape-memory main support 3502, may facilitate such motion. In certain embodiments, backward (clockwise) motion is inhibited by the engagement of the pawl 3508 against the back wall of the start guide channel 3518. For example, in the illustrated embodiment, backward motion is inhibited due to the substantially perpendicular angle of the back wall of the start guide channel 3518 (and detents), while forward motion is facilitated by the increasing slope of the front wall of the start guide channel 3518 (and detents). In certain embodiments, other mechanisms may be used to facilitate and/or inhibit movement of the pawl 3508. The illustrated embodiment shows the sleeve 3530 affixed to the main support 3502 and positioned to expose the pawl 3508. In certain embodiments, the sleeve 3530 may cover the pawl 3508 in order to protect the adjustment mechanism. In certain embodiments, the sleeve 3530 may cover the pawl 3508 in order to ensure that the pawl 3508 does not disengage from the start guide channel 3518.

FIG. 38B illustrates a side view of an embodiment of the implant of FIG. 35A in its minimum diameter configuration. The implant 3500 comprises the main support 3502, the return spring 3504, the containment sleeve 3530, the pawl 3508, the pawl spring 3522, a plurality of ratchet detents 3510, and a return ratchet detent 3514. The forward end of the return ratchet detent 3514 is slanted to facilitate entry of the pawl 3508 into a return channel 3602 should the pawl 3508 be advanced to rest against the return ratchet detent 3514, as described above. In the embodiment illustrated, the pawl 3508 has been advanced so that it rests against the forward edge of the last ratchet detent 3510 before the return detent 3514. The pawl 3508 stably rests against the forward end of the ratchet detent 3510 and is prevented from moving in the backward direction, as described above. In certain embodiments, the illustrated configuration may be the smallest radius, smallest length, and/or smallest arc length of the implant 3500.

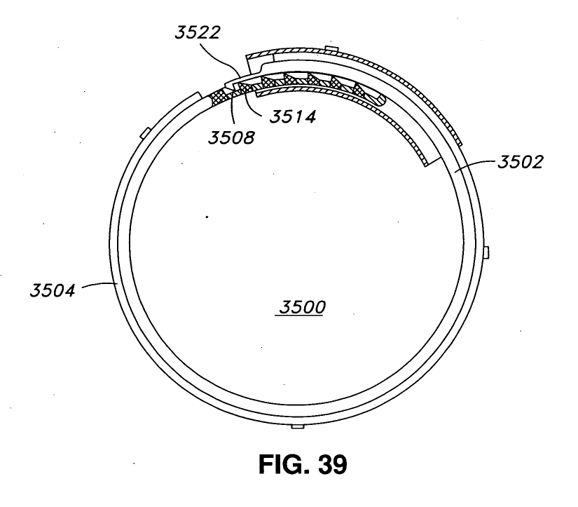

FIG. 39 illustrates a side view of the implant of FIG. 35A in its unstable minimum diameter return configuration, just prior to removing the actuation energy from the main support 3502. While activation energy is applied to the main support 3502, the pawl 3508 may remain in the temporary adjusted state, as illustrated. However, once the energy has been removed from the implant, the return spring 3504 governs the force balance in the implant 3500 causing the pawl 3508 to be forced backward against the return detent 3514. Since the return detent 3514 has an angled forward face, as described above, the pawl 3508, or the pawl spring 3522 to which the pawl 3508 is affixed, may deflect the pawl 3508 sideways into the return channel 3602, as described above. Once the pawl 3508 has been deflected into the return channel 3602, the implant 3500 will revert to the start configuration illustrated in FIG. 38A after the pawl 3508 follows a return groove to come to a stable rest in the start guide channel 3518.

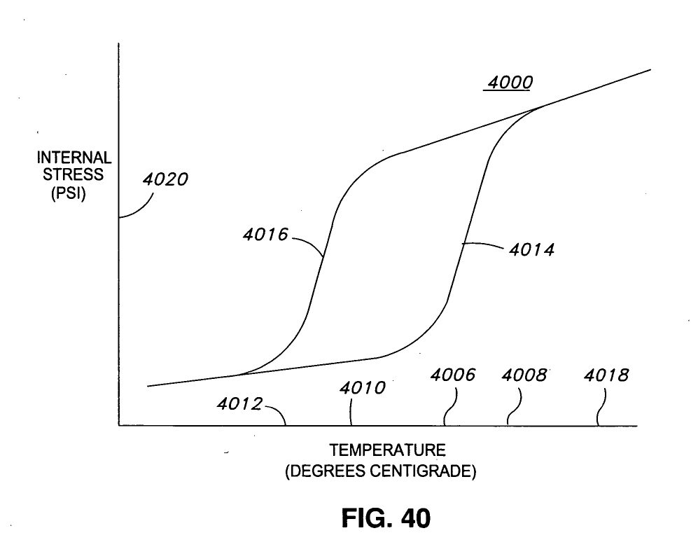

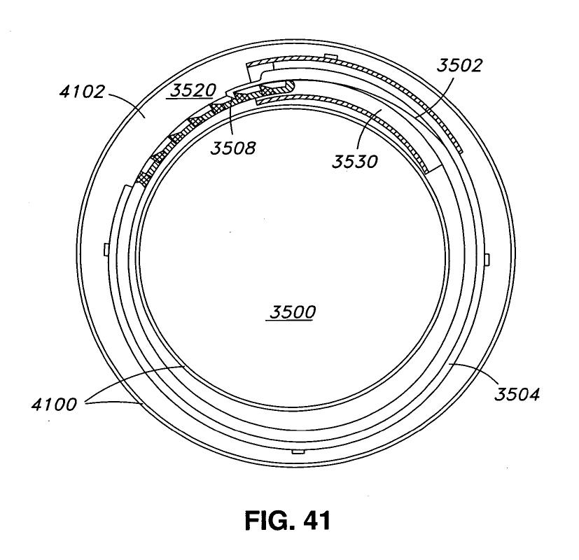

FIG. 40 illustrates a plot 4000 of internal stress 4020 versus temperature 4018 for a shape-memory material which can be used to power certain embodiments of the implant 3500 illustrated in FIG. 35. In certain embodiments, the shape-memory material may be integral to the main support 3502 of the implant 3500. In certain embodiments, the shape-memory material may be a separate material affixed to the main support 3502.

The plot 4000 includes the x-axis 4018, the y-axis 4020 and illustrates the relationship between temperature 4018 on the x-axis 4018 and stress 4020 which is plotted as the y-axis 4020 wherein the increasing temperature curve 4014 is differentiated from the decreasing temperature curve 4016. The increasing temperature curve 4014 is different from the decreasing temperature curve 4016 due to hysteresis effects in the material that cause it to behave differently depending on whether the temperature is increasing or decreasing.

The plot 4000 further includes the transition points which are plotted at specified points on the temperature x-axis 4018. These transition points include the Aₛ temperature 4006, the A_{f} temperature 4008, the Mₛ temperature 4010, and the M_{f} temperature 4012. In certain embodiments, the M_{f} temperature may be greater than a maximum body temperature. Body temperature is normally around 37 degrees centigrade, but may rise as high as 40 to 42 degrees centigrade due to a high fever. Thus, in certain embodiments, M_{f} is at or above 40 degrees centigrade. In certain embodiments, Aₛ is approximately 72° C., A_{f} is approximately 88° C., Mₛ is approximately 56° C., and M_{f} is approximately 40° C. In certain embodiments, other temperatures may be used. In certain embodiments, shape-memory materials may be insulated from the body tissues so that local overheating of tissue does not occur when shape-memory materials increase in temperature.

FIG. 41 illustrates an embodiment of the implant 3500 of FIG. 35A further comprising an external layer 4100. The implant 3500 comprises the main support 3502, the return spring 3504, the shroud 3530, the pawl 3508, and the ratchet assembly 3520. In certain embodiments, the external layer 4100 may be a coating. The coating may be applied in certain embodiments according to the coating techniques described above. In certain embodiments, the external layer 4100 can swell following implantation. In certain embodiments, the external layer 4100 further comprises a compressible region 4102 surrounding the ratchet assembly 3520. In certain embodiments, the external layer 4100 is fabricated from hydrophilic hydrogel or other water-swellable polymer capable of drawing water from surrounding tissue or fluids and incorporating the water into its structure. In certain embodiments, suitable hydrogels can comprise materials such as, but not limited to, polyethylene glycol, Poly 2-Hydroxyethylmethacrylate (pHEMA), and the like. When the hydrogel layer 4100 absorbs water, its volume increases substantially, thus increasing a width dimension of the implant 3500. The external layer 4100 may be dried prior to implantation; consequently, in certain embodiments, it 4100 may be relatively thin when dry. In certain embodiments, the external layer 4100 may be somewhat hard and inflexible when dry. In certain embodiments, the external layer 4100 is dried during manufacturing of the implant 3500 and/or layer 4100.

Upon implantation, the external layer 4100 may swell substantially by drawing water from blood or tissue within which it is implanted. Following absorption, the external layer 4100 may be soft, and capable of elastomeric expansion or compression. For example, the hydrogel layer 4100 may compress circumferentially with a corresponding thickness increase should the implant 3500 decrease in diameter, causing a resulting decrease in the circumference of the implant 3500.

The swellable layer 4100 is advantageous in that it allows the implant to be inserted using minimally invasive techniques due to a minimum profile configuration. Following implantation, the hydrogel layer/coating on the implant may swell in volume, generating an increased footprint, effective strain relief, and minimum force per unit area (i.e., pressure) exerted on the underlying tissue. In certain embodiments, an external layer 4100 using a hydrogel coating may be configured to increase its thickness by a factor of two. In certain embodiments, an external layer 4100 using a hydrogel coating may be configured to increase its thickness by a factor of five. In certain embodiments, an external layer 4100 using a hydrogel coating may be configured to increase its thickness by a factor of ten. For example, a coating that is 0.5 mm thick may swell to become 5 mm thick or thicker (a volume-factor increase of ten). In certain embodiments, an external layer 4100 using a hydrogel coating may be configured to increase its thickness by a factor of more than ten. In certain embodiments, an external layer 4100 using a hydrogel coating may be configured to increase its thickness by a factor of less than two. In certain embodiments, an external layer 4100 using a hydrogel coating may be configured to increase its thickness by a factor of up to 3500. In certain embodiments, a large factor increase may cause a loss of internal strength due to the large proportion of water to polymer in the structure.

An external layer 4100 may be selectively applied and/or configured so as to be thicker in some regions of the implant 3500 and thinner in other regions of the implant 3500. For example, the implant 3500 may be made to achieve increased width, following swelling, with a substantially low amount of thickness increase. In certain embodiments, the external layer 4100 may comprise active pharmacological agents such as, but not limited to, antimicrobial agents, antibiotics, antiviral agents, tissue growth inhibitors, anti-cancer drugs, anti-thrombogenic agents, thrombogenic agents, thrombolytic agents, and the like. In certain embodiments, the external layer 4100 may provide for time release of the agents or for permanent retention of the agents.

In certain embodiments, the implant 3500 may further be covered with a non-swelling coating comprising materials such as, but not limited to, silicone elastomer, polyester velour, PTFE, or the like, either as solid materials, foams, or fabrics such as knits, velour, or weaves.

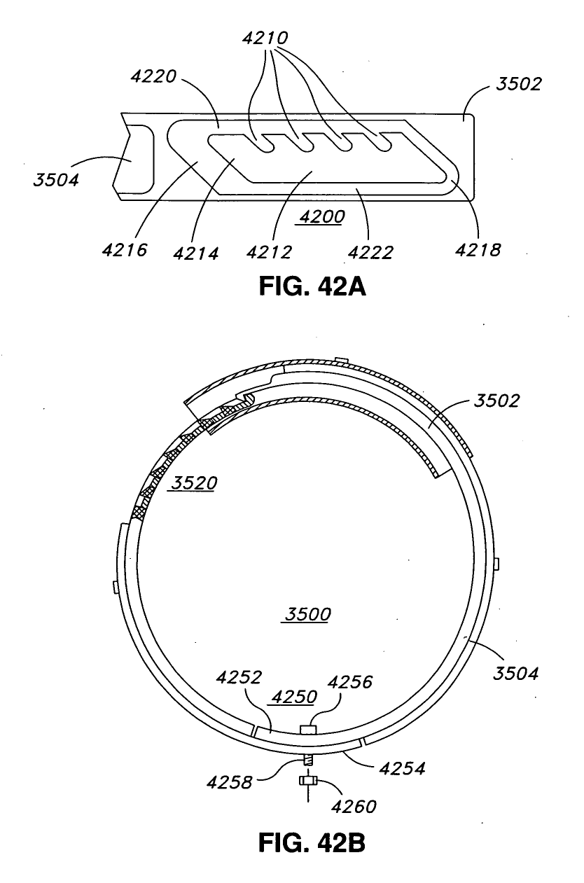

FIG. 42A illustrates a face on view of an embodiment of a ratchet mechanism 4200 of the implant 3500 of FIG. 35A wherein the ratchet mechanism 4200 operates in a single plane. In certain embodiments, the ratchet mechanism 4200 may operate in more than one plane. In certain embodiments, the ratchet mechanism 4200 may be affixed or integral to the main support 3502 of an implant as described in FIG. 35. In the illustrated embodiment, the pawl 3508 of FIG. 35A is replaced by a pawl pin or mushroom capped pin (not illustrated) that engages with the track of the ratchet 4200.

The ratchet mechanism 4200 comprises a start track 4218, a return track 4222, a forward track 4220, a plurality of ratchet slots 4210, a central bulkhead 4212, a return ratchet guide 4214 and a return end slot 4216. Also shown is the edge of the return spring 3504. The pawl pin is biased in the direction of the return track 4222 by the return spring 3504 so as to engage the nearest ratchet slot 4210 after its current position when advanced forward due to the temporary application of activation energy to the main support 3502. Whereas the embodiment illustrated in FIG. 42A may be adjusted by horizontal movement causing engagement and disengagement of its pawl to the ratchet mechanism 4200, the embodiment illustrated in FIG. 35A may be adjusted by both horizontal and vertical movement causing engagement and disengagement of its pawl 3508 to the ratchet mechanism 3520. In substantially all other aspects, this embodiment can be substantially similar to the embodiment illustrated in FIG. 35.

FIG. 42B illustrates an embodiment of the implant 3500 of FIG. 35A further comprising a separable region 4250 opposite the ratchet mechanism 3520. In certain embodiments, the separable region 4250 comprises a first end 4252, a second end 4254, a first fastener 4256 and a second fastener 4260. In certain embodiments, the separable region is closed by passing the first fastener 4256 through a hole in the second end 4254 and applying the second fastener 4260 to the first fastener 4256 so as to secure the connection. For example, the first fastener 4256 may be a bolt and the second fastener 4260 may be a nut. In certain embodiments, other fasteners may be used, such as a screw, clip, button, bayonet mount, quick connect, or the like.

In certain embodiments, the separable region 4250 may be a simple belt buckle and belt end, or other connector suitable for a band-like structure. Separable embodiments of the implant 3500 may be suitable for use in applying the implant 3500 around a structure while leaving the ratchet mechanism 3520 in tact and not disengaged at any point. In certain embodiments, the ratchet mechanism 3520 may have a separable or removable casing (not illustrated) so as to permit opening of the ratchet mechanism 3520, and to permit the implant 3500 to be placed around an object and then be closed. Following placement, the casing 3530 is reassembled to constrain the ratchet mechanism 3520 from disengagement.

While certain aspects and embodiments of the invention have been described, these have been presented by way of example only, and are not intended to limit the scope of the invention. Indeed, the novel methods and systems described herein may be embodied in a variety of other forms without departing from the spirit thereof. The accompanying claims and their equivalents are intended to cover such forms or modifications as would fall within the scope and spirit of the invention.

What is claimed is:

1. An adjustable implant, configured to be implanted within or at least partially around an outer surface of a stomach or esophagus, comprising:
a ratchet; and
an elongate band comprising a shape-memory material, wherein a first end and a second end of the elongate band are configured to couple to the ratchet, such that the band and the ratchet form an assembly having a loop configuration;
wherein activation of the shape-memory material adjusts the band from a first length to a second length as the ratchet permits movement in a first direction of the first end relative to the second end, changing a circumference of the loop configuration.

2. The adjustable implant of claim 1, wherein the elongate band further comprises a pawl spring that comprises the shape-memory material.

3. The adjustable implant of claim 1, wherein the ratchet permits movement in a second direction of the first end relative to the second end, changing the circumference of the loop.

4. The adjustable implant of claim 1, wherein the ratchet comprises a pawl and a detent.

5. The adjustable implant of claim 1, the assembly being configured to be formed as a loop within or around a portion of the stomach or esophagus, and wherein the elongate band is configured to change a dimension of a lumen of the portion of the stomach or esophagus by adjusting the circumference of the loop.

6. The adjustable implant of claim 1, the assembly being configured to encircle at least partially a portion of the stomach.

7. The adjustable implant of claim 1, wherein the assembly is configured to decrease the circumference of the loop.

8. The adjustable implant of claim 1,
wherein the ratchet further comprises a plurality of detents;
wherein the elongate band comprises a pawl at the second end of the elongate band;
and wherein the elongate band is configured to decrease or increase the circumference of the loop by drawing a detent past the pawl when the shape-memory material is activated.

9. The adjustable implant of claim 1, wherein the elongate band is configured to be implanted around a portion of the stomach to form a gastric pouch, and wherein the elongate band is configured to change a size of a lumen in the gastric pouch by adjusting, a circumference of the loop.

10. The adjustable implant of claim 1, wherein the elongate band comprises a polymer.

11. The adjustable implant of claim 1, wherein the shape-memory material comprises at least one of a metal, a metal alloy, a nickel titanium alloy, and a shape-memory polymer.

12. The adjustable implant of claim 1, wherein the shape-memory material comprises at least one of Fe—C, Fe—Pd, Fe—Mn—Si, Co—Mn, Fe—Co—Ni—Ti, Ni—Mn—Ga, Ni₂MnGa, and Co—Ni—Al.

13. The adjustable implant of claim 1, wherein the elongate band is configured to detach into a first band portion and a second band portion.

14. The adjustable implant of claim 1, further comprising a hydrophilic material substantially coating at least a portion of the implant.

15. The adjustable implant of claim 14, wherein the hydrophilic material comprises at least one of polyethylene glycol and Poly 2-Hydroxyethylmethacrylate.

16. The adjustable implant of claim 14, wherein an external layer having a varying thickness comprises the hydrophilic material.

17. The implantable device of claim 1, further comprising a restraint that at least partially encloses at least a portion of the ratchet and is configured to prevent or reduce at least one of (1) the first end of the elongate band from uncoupling from the ratchet, and (2) encroachment by tissue into a region at the first end.

18. The adjustable implant of claim 1, further comprising a spring return mechanism coupled to the elongate band.

19. An adjustable implant configured to be implanted around an outer surface of a stomach or esophagus, comprising:
encircling means for at least partially surrounding the stomach or esophagus, the encircling means comprising a shape-memory material; and
ratchet means for permitting movement in a first direction of a first end of the encircling means relative to a second end of the encircling means;
wherein activation of the shape-memory material adjusts the encircling means from a first length to a second length as the ratchet means permits the movement of the first end relative to the second end, changing a circumference of the encircling means.

20. The adjustable implant of claim 19, further comprising an activation means configured to provide an activation energy to the shape-memory material.

21. A method, for treating obesity, the method comprising the steps of:
placing an adjustable implant within or around a patient's stomach or esophagus, the adjustable implant comprising:
a ratchet; and
an elongate band comprising a shape-memory material, wherein a first end and a second end of the elongate band are configured to couple to the ratchet, such that the band and the ratchet form an assembly having a loop configuration;
wherein activation of the shape-memory material adjusts the band from a first length to a second length as the ratchet permits movement in a first direction of the first end relative to the second end, changing a circumference of the loop configuration;
applying an activation energy to the shape-memory material; and
transforming the shape-memory material from a first configuration to a second configuration, thereby changing the circumference of the loop configuration.

22. The method of claim 21, wherein the ratchet comprises a plurality of serially arranged detents, and changing the circumference of the loop configuration comprises moving the first end of the elongate band from a first position, at one of the plurality of detents, to a second position, at another of the plurality of detents.

23. The method of claim 21, wherein the implant further comprises a spring configured to expand the circumference of the loop configuration to a maximum circumference.

24. The method of claim 21, wherein the implant further comprises a hydrophilic coating that substantially coats at least a portion of the implant.

25. The method of claim 21, wherein the implant comprises a pre-implantation shape and a post-implantation shape, further comprising:
laparoscopically inserting the implant in the pre-implantation shape into the patient, so as to facilitate having the implant assume the post-implantation shape around the stomach or esophagus.

26. The method of claim 21, wherein the implant comprises a pre-implantation shape and a post-implantation shape, further comprising:
endoscopically inserting the implant in the pre-implantation shape into the patient, so as to facilitate having the implant assume the post-implantation shape within the stomach or esophagus.

27. The method of claim 21, wherein the shape-memory material comprises. at least one of a metal, a metal alloy, a nickel titanium alloy, and a shape-memory polymer.

28. The method of claim 27, wherein the shape-memory material comprises at least one of Fe—C, Fe—Pd, Fe—Mn—Si, Co—Mn, Fe—Co—Ni—Ti, Ni—Mn—Ga, Ni₂MnGa, and Co—Ni—Al.

29. The method of claim 21, wherein the activation energy comprises at least one of magnetic resonance imaging energy, high-intensity focused ultrasound energy, radio frequency energy, x-ray energy, microwave energy, light energy, electric field energy, magnetic field energy, inductive heating, and conductive heating.
